(19) United States
(12) Patent Application Publication
(10) Pub. No.: US 2015/0005186 A1
(43) Pub. Date: Jan. 1, 2015

(54) METHODS AND DEVICES FOR CLASSIFYING AND MANAGING AUTOIMMUNE AND INFLAMMATORY CONDITIONS

(71) Applicant: Jing-Feng HUANG, San Diego, CA (US)

(72) Inventor: Jing-Feng HUANG, San Diego, CA (US)

(21) Appl. No.: 14/318,556

(22) Filed: Jun. 27, 2014

Related U.S. Application Data
(60) Provisional application No. 61/840,450, filed on Jun. 27, 2013.

(51) Int. Cl. G01N 33/68

(52) U.S. Cl. CPC G01N 33/6863; G01N 2800/16
USPC 506/9

(57) ABSTRACT

Devices and methods useful for biomarker-based diagnosis of immunopathological mechanisms involved in autoimmune or inflammatory diseases are described.

### RELATED APPLICATION

This application claims the benefit of and priority to U.S. provisional patent application Ser. No. 61/840,450, filed 27 Jun. 2013 (attorney docket no. JFG-0030-PV2) and entitled, “Methods and Devices for Classifying and Managing Autoimmune and Inflammatory Conditions,” the contents of which are hereby incorporated by reference for any and all purposes.

### TECHNICAL FIELD

This invention concerns devices, kits, and methods for diagnosing and differentiating subtypes of immunopathological mechanisms involved in autoimmune and inflammatory diseases using biomarkers; methods of diagnosis, monitoring, and screening using such biomarkers; and devices (e.g., kits) for performing such methods; methods for managing autoimmune and inflammatory diseases caused by mechanisms; as well as to ways of using data and information generated through the use of such devices and methods.

### BACKGROUND OF THE INVENTION

### 1. Introduction

The following description includes information that may be useful in understanding the present invention. It is not an admission that any such information is prior art, or relevant, to the presently claimed inventions, or that any publication specifically or implicitly referenced is prior art.

### 2. Background

The human immune system is complex. The antigen (Ag) non-specific innate immunity and Ag-specific adaptive immunity arms synergize to defend against invading pathogens and respond to tissue damage and host-derived stress signals. The intrinsic complexity of the immune system renders it prone to dysfunction, leading to cancer, autoimmunity, chronic inflammation, chronic infections, and allergy.

Even though autoimmunity, allergy, and many inflammatory diseases are chronic and involve remissions and relapses, reliable biomarkers of disease activity and predictors of flares or acute episodes are missing. Pathogenic mechanisms of and inflammatory pathways involved in many of these diseases are not well characterized, either. Furthermore, many of these diseases continue to be treated in a non-specific manner with drugs that suppress broad inflammatory cascades.

Systemic Lupus Erythematosus (SLE) is a systemic autoimmune disease, with autoantibody production and immune complex deposition as the hallmark features. SLE patients present with the widest array of clinical manifestations, including those whose disease affects multiple organs, including the skin, kidneys, blood vessels, CNS, etc. Sjogren's syndrome (SS) is a systemic autoimmune disease primarily characterized by chronic inflammation of the exocrine glands, in particular the salivary and lacrimal glands, and can develop alone or in association with other autoimmune diseases such as SLE and rheumatoid arthritis (RA). Similar to SLE, SS is also marked with presence of autoantibodies, anti-SS-A and anti-SS-B antibodies. In SS patients with ocular involvement, destruction and malfunction of the lachrymal gland, the exocrine gland responsible for secreting aqueous components of the tear film in human, leads to significant reduction in tear production and ocular dryness, which leads to development of dry eye condition.

Dry eye is a common ocular surface inflammatory disease that significantly affects quality of life. Dry eye disease includes a heterogeneous group of chronic conditions, each of which could associate with a different cause, such as systemic autoimmunity in SS leading to ocular dryness, or obstruction of the meibomian gland in meibomian gland dysfunction (MGD), which leads to changes in the composition and quality of the tear film and ocular surface damage. All such conditions are characterized, however, by the development of ocular symptoms, accompanied by various clinical signs and pathological changes, including reduced tear production, unstable tear layer, irregular corneal surface, loss of goblet cells on the conjunctival epithelia, hyperplasia and sensitization of corneal nerve endings, and development of corneal epitheliopathy.

Non-infectious uveitis is a group of intraocular inflammatory conditions and is a significant cause of visual disability worldwide.

The success of different modalities of TNF-alpha blockers in treating psoriasis, RA, psoriatic arthritis, and spondyloarthropaties demonstrates the significance and beneficial effect of blocking a single cytokine in several diseases with common as well as unique clinical manifestations. Blockers of other individual cytokines, such as IL-6 or IL-1, also show certain levels of efficacy in RA. However, the heterogeneous response among patients to drugs blocking TNF-alpha, IL-6, or IL-1 indicates the need for better understanding of the different immunopathological mechanisms involved in these conditions, their association with disease activities or stages, and disease subtypes that contribute to patient heterogeneity.

In Sjogren's syndrome patients, activation of the IL-23-Th17 pathway, abnormal expression of B-cell-activating factor (BAFF), and up-regulation of type I interferon-regulated genes have all been described. Several cytokines, including BLys (BAFF), IL-6, IL-17, IL-18, type I interferon (IFN), and TNF-α, have all been implicated in the pathogenesis of SLE. CD4+ T cells, in particular Th1 and Th17, and proinflammatory cytokines IFN-γ, IL-1, IL-17, IL-6, and IL-8, are thought to be associated with dry eye disease pathogenesis. In uveitis, the IL-23-Th17 pathway and IL-1 have been implicated.

IL-1beta, IL-1alpha, and IL-18 are members of the IL-1 family. IL-1 receptor antagonist (IL-1Ra) is also a member of the IL-1 family, but it is an endogenous antagonist of IL-1beta and IL-1alpha because it binds to IL-1 receptor but does not trigger signaling.

There are three main groups of CD4+ effector T cells and their associated cytokines: Th1 cells and Th1 associated IFNgamma and IL-2, Th2 cells and Th2 associated IL-10, -3, -4, -5, -13 and GM-CSF, and Th17 cells and Th17 associated IL-17A. These T cell cytokines can also be produced by the three corresponding groups of innate lymphoid cells (ILCs). The type 1 and type 17 are proinflammatory. Pathogenic Th17 is thought to be an important cell type and play an important role in driving autoimmunity in a number of autoimmune conditions.

Type I IFN is induced upon viral infection or by immune complexes containing RNA and by RNA-associated autoantigens such as in SLE and SS. In addition to antiviral activities, type I IFN, together with IL-6, also contribute to increase autoantibody secretion and B cells differentiation into plasma cells. Type I IFN was shown to have differential effects on gene expression of different members of the IL-1 family, as it suppresses gene expression of IL-1a and 1b, and IL-1R1 and up-regulate expression of IL-1Ra, IL-1R2, and IL-18. The net effect is the dampening of the IL-1 pathway and the enhancement of the IL-18 pathway. IL-1 activity supports Th17 responses and IL-17 production, while IL-18 activity is linked with IFN-γ production.

Unlike RA, psoriasis, psoriatic arthritis, and spondyloarthropaties, currently there are limited options for a safe and effective treatment for SLE, SS, uveitis, or dry eye. In addition, diagnosing SS, uveitis, and, in particular, SLE, and measuring disease activity, represent a challenge to doctors. Thus, there is significant need to develop efficient and objective systems to assess global disease activity in these conditions.

However, with the complexity, redundancy, and inter-regulation of the immune system, a standard reductionist approach and experiments are insufficient to strike progress in the understanding of complex human systems. Thus, there is significant value in taking novel strategies, such as systems analysis, to analyze large data sets to characterize molecular mechanisms involved in immune-mediated diseases, identify patient subtypes, extract corresponding biomarker signatures for patient stratification (classification), prognosis, and treatment management, and to identify specific targets for development of therapies.

### 3. Definitions

Before Describing the Instant Invention in Detail, Several Terms Used in the Context of the Present Invention will be defined. In addition to these terms, others are defined elsewhere in the specification, as necessary. Unless otherwise expressly defined herein, terms of art used in this specification will have their art-recognized meanings.

The term “risk” relates to the possibility or probability of a particular event occurring either presently, or, at some point in the future. “Risk stratification” refers to an arraying of known clinical risk factors to allow physicians to classify patients into a low, moderate, high or highest risk of developing of a particular disease, disorder, or condition.

“Diagnosing” includes determining, monitoring, confirmation, subclassification, and prediction of the relevant disease, complication, or risk. “Determining” relates to becoming aware of a disease, complication, risk, or entity (e.g., biomarker). “Monitoring” relates to keeping track of an already diagnosed disease, complication, or risk factor, e.g., to analyze the progression of the disease or the influence of a particular treatment on the progression of disease or complication. “Confirmation” relates to the strengthening or substantiating of a diagnosis already performed using other indicators or markers. “Classification” or “subclassification” relates to further defining a diagnosis according to different subclasses of the diagnosed disease, disorder, or condition, e.g., defining according to mild, moderate, or severe forms of the disease or risk. “Prediction” relates to prognosing a disease, disorder, condition, or complication before other symptoms or markers have become evident or have become significantly altered.

A “subject” is a member of any animal species, preferably a mammalian species, optionally a human. Thus, the methods and compositions described herein are applicable to both human and veterinary disease. Further, while a subject is preferably a living organism, the invention described herein may be used in post-mortem analysis as well. Preferred subjects are humans, and most preferably “patients,” which as used herein refers to living humans that are receiving medical care for a disease or condition. This includes persons with no defined illness who are being investigated for signs of pathology. The subject can be an apparently healthy individual, an individual suffering from a disease, or an individual being treated for a disease. A “reference subject” or “reference subjects” is/are an individual or a population that serves as a reference against which to assess another individual or population with respect to one or more parameters.

The term “normal” or “clinically normal” means the subject has no known or apparent or presently detectable disease or dysfunction and no detectable increase in biomarkers associated with autoimmune and/or inflammatory diseases.

“Biological samples” that can be assayed using the methods of the present invention include biological fluids, such as whole blood, serum, plasma, tear, saliva, synovial fluid, cerebrospinal fluid, bronchial lavage, ascites fluid, bone marrow aspirate, pleural effusion, urine, as well as tumor tissue or any other bodily constituent or any tissue culture supernatant that could contain the analyte of interest. Samples can be obtained by any appropriate method known in the art.

An “analyte” refers to the substance to be detected, which may be suspected of being present in the sample (i.e., the biological sample). The analyte can be any substance for which there exists a naturally occurring specific binding partner or for which a specific binding partner can be prepared. Thus, an analyte is a substance that can bind to one or more specific binding partners in an assay.

A “binding partner” is a member of a binding pair, i.e., a pair of molecules wherein one of the molecules binds to the second molecule. Binding partners that bind specifically are termed “specific binding partners.” In addition to antigen and antibody binding partners commonly used in immunoassays, other specific binding partners can include biotin and avidin (or streptavidin), carbohydrates and lectins, nucleic acids with complementary nucleotide sequences, effector and receptor molecules, cofactors and enzymes, enzyme inhibitors and enzymes, and the like. Furthermore, specific binding partners can include partner(s) that is/are analog(s) of the original specific binding partner, for example, an analyte-analog. Immunoreactive specific binding partners include antigens, antigen fragments, antibodies and antibody fragments, both monoclonal and polyclonal, and complexes thereof, including those formed by recombinant DNA methods.

As used herein, the term “epitope” or “epitopes,” or “epitopes of interest” refer to a site(s) on any molecule that is recognized and is capable of binding to a complementary site(s) on its specific binding partner. The epitope-bearing molecule and specific binding partner are part of a specific binding pair. For example, an epitope can be a polypeptide, protein, hapten, carbohydrate antigen (such as, but not limited to, glycolipids, glycoproteins or lipopolysaccharides) or polysaccharide and its specific binding partner, can be, but is not limited to, an antibody. Typically an epitope is contained within a larger molecular framework (e.g., in the context of an antigenic region of a protein, the epitope is the region or fragment of the protein having the structure capable of being bound by an antibody reactive against that epitope) and refers to the precise residues known to contact the specific binding partner. As is known, it is possible for an antigen or antigenic fragment to contain more than one epitope.

As used herein, “specific” or “specificity” in the context of an interaction between members of a specific binding pair (e.g., an antigen and antibody) refers to the selective reactivity of the interaction. The phrase “specifically binds to” and analogous terms thereof refer to the ability of autoantibodies to specifically bind to (e.g., preferentially react with) an endogenous antigen and not specifically bind to other entities. Antibodies (including autoantibodies) or antibody fragments that specifically bind to an endogenous antigen correlated with dry eye disease can be identified, for example, by diagnostic immunoassays (e.g., radioimmunoassays (“RIA”) and enzyme-linked immunosorbent assays (“ELISAs”), surface plasmon resonance, or other techniques known to those of skill in the art. In one embodiment, the term “specifically binds” or “specifically reactive” indicates that the binding preference (e.g., affinity) for the target analyte is at least about 2-fold, more preferably at least about 5-fold, 10-fold, 100-fold, 1.000-fold, a million-fold or more over a non-specific target molecule (e.g., a randomly generated molecule lacking the specifically recognized site(s)).

An antigen, biomarker, or other analyte “correlated” or “associated” with a disease, particularly dry eye disease, refers to a biomarker or other analyte that is positively correlated with the presence or occurrence of dry eye disease generally or a specific dry eye disease, as the context requires. In general, an “antigen” is any substance that exhibits specific immunological reactivity with a target antibody. Suitable antigens, particularly biomarkers, may include, without limitation, molecules comprising at least one antigenic epitope capable of interacting specifically with the variable region or complementarity determining region (CDR) of an antibody or CDR-containing antibody fragment. Antigens typically are naturally occurring or synthetic biological macromolecules such as a protein, peptide, polysaccharide, lipids, or nucleic acids, or complexes containing these or other molecules.

As used herein with reference to a disease-associated antigens (or other analytes correlated with dry eye disease), the term “elevated level” refers to a level in a sample that is higher than a normal level or range, or is higher that another reference level or range (e.g., earlier or baseline sample). The term “altered level” refers to a level in a sample that is altered (increased or decreased) over a normal level or range, or over another reference level or range (e.g., earlier or baseline sample). The normal level or range for a particular biomarker is defined in accordance with standard practice. Because the levels of biomarkers in some instances will be very low, a so-called altered level or alteration can be considered to have occurred when there is any net change as compared to the normal level or range, or reference level or range that cannot be explained by experimental error or sample variation. Thus, the level measured in a particular sample will be compared with the level or range of levels determined in similar samples of normal tissue. In this context, “normal tissue” is tissue from an individual with no detectable dry eye pathology, and a “normal” (sometimes termed “control”) patient (i.e., subject) or population is one that exhibits no detectable pathology. The level of an analyte is said to be “elevated” where the analyte is normally undetectable (e.g., the normal level is zero, or within a range of from about 25 to about 75 percentiles of normal populations), but is detected in a test sample, as well as where the analyte is present in the test sample at a higher than normal level.

An “array” refers a device consisting of a substrate, typically a solid support having a surface adapted to receive and immobilize a plurality of different protein, peptide, and/or nucleic acid species (i.e., capture or detection reagents) that can used to determine the presence and/or amount of other molecules (i.e., analytes) in biological samples such as blood. A “microarray” refers to an array wherein the different detection reagents disposed on the substrate.

The term “solid phase” refers to any material or substrate that is insoluble, or can be made insoluble by a subsequent reaction. A solid phase can be chosen for its intrinsic ability to attract and immobilize a capture or detection reagent. Alternatively, a solid phase can have affixed thereto a linking agent that has the ability to attract and immobilize a capture agent. The linking agent can, for example, include a charged substance that is oppositely charged with respect to the capture agent itself or to a charged substance conjugated to the capture agent. In general, a linking agent can be any binding partner (preferably specific) that is immobilized on (said to be “attached to”) a solid phase and that has the ability to immobilize a desired capture or detection reagent through a binding or other associative reaction. A linking agent enables the indirect binding of a capture agent to a solid phase material before the performance of an assay or during the performance of an assay. The solid phase can, for example, be plastic, derivatized plastic, magnetic or non-magnetic metal, glass or silicon, including, for example, a test tube, microtiter well, sheet, bead, microparticle, chip, and other configurations known to those of ordinary skill in the art.

As used herein, term “microparticle” refers to a small particle that is recoverable by any suitable process, e.g., magnetic separation or association, ultracentrifugation, etc. Microparticles typically have an average diameter on the order of about 1 micron or less.

A “capture” or “detection” agent or reagent refers to a binding partner that binds to an analyte, preferably specifically. Capture or detection reagents can be attached to or otherwise associated with a solid phase.

The term “labeled detection agent” refers to a binding partner that binds to an analyte, preferably specifically, and is labeled with a detectable label or becomes labeled with a detectable label during use in an assay.

A “detectable label” includes a moiety that is detectable or that can be rendered detectable. With reference to a labeled detection agent, a “direct label” is a detectable label that is attached, by any means, to the detection agent, and an “indirect label” is a detectable label that specifically binds the detection agent. Thus, an indirect label includes a moiety that is the specific binding partner of a moiety of the detection agent. Biotin and avidin are examples of such moieties that can be employed, for example, by contacting a biotinylated antibody with labeled avidin to produce an indirectly labeled antibody.

The term “indicator reagent” refers to any agent that is contacted with a label to produce a detectable signal. Thus, for example, in conventional enzyme labeling, an antibody labeled with an enzyme can be contacted with a substrate (the indicator reagent) to produce a detectable signal, such as a colored reaction product.

An “antibody” refers to a protein consisting of one or more polypeptides substantially encoded by immunoglobulin genes or fragments of immunoglobulin genes. This term encompasses polyclonal antibodies, monoclonal antibodies, and fragments thereof, as well as molecules engineered from immunoglobulin gene sequences. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. Antibodies are generally found in bodily fluids, mainly blood.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain (VL)” and “variable heavy chain (VH)” refer to these light and heavy chains, respectively.

Antibodies exist as intact immunoglobulins or as a number of well-characterized fragments produced by digestion with various peptidases. Thus, for example, pepsin digests an antibody below the disulfide linkages in the hinge region to produce F(ab′)₂, a dimer of Fab which itself is a light chain joined to VH-CH1 by a disulfide bond. The F(ab′)₂ may be reduced under mild conditions to break the disulfide linkage in the hinge region thereby converting the (Fab′)₂ dimer into a Fab′ monomer. The Fab′ monomer is essentially a Fab with part of the hinge region. While various antibody fragments are defined in terms of the digestion of an intact antibody, one of skill will appreciate that such Fab′ fragments may be synthesized de novo either chemically or by utilizing recombinant DNA methodology. Thus, in the context of the invention the term “antibody” also includes antibody fragments either produced by the modification of whole antibodies or synthesized de novo using recombinant DNA methodologies. Antibodies include single chain antibodies (antibodies that exist as a single polypeptide chain), single chain Fv antibodies (sFv or scFv), in which a variable heavy and a variable light chain are joined together (directly or through a peptide linker) to form a continuous polypeptide. The single chain Fv antibody is a covalently linked VH-VL heterodimer that may be expressed from a nucleic acid including VH- and VL-encoding sequences either joined directly or joined by a peptide-encoding linker. While the VH and VL are connected to each as a single polypeptide chain, the VH and VL domains associate non-covalently. The scFv antibodies and a number of other structures convert the naturally aggregated, but chemically separated, light and heavy polypeptide chains from an antibody V region into a molecule that folds into a three dimensional structure substantially similar to the structure of an antigen-binding site are known to those of skill in the art.

A “panel” refers to a group of two or more distinct molecular species that have shown to be indicative of or otherwise correlated with a particular disease or health condition. Such “molecular species” may be referred to as “biomarkers”, with the term “biomarker” being understood to mean a biological molecule the presence or absence of which serves as an indicator of a particular biological state, for example, the occurrence (or likelihood of the occurrence) of dry eye disease in a subject. In other words, a biomarker is a characteristic that can objectively measured and evaluated as an indicator of normal biologic processes, pathogenic processes, or pharmacologic responses to a therapeutic intervention. In the context of the invention an “assay panel” or “array panel” refers to an article, typically a solid phase substrate, having a panel of capture reagents associated therewith (typically by immobilization), wherein at least one of the capture reagents is specifically reactive with a biomarker associated with dry eye disease. In some embodiments, an assay panel includes 2, 3, 4, 5, 6, 7, 8, 9, 10, 11, 12, 13, 14, 15, 16, 17, 18, 19, 20, or more (e.g., 25, 30, 35, 40, 50, 75, 100, 150, 200, 250, 500, etc., including any integer, or range of integers from 1 to 500) different detection reagents, alone or combination with other detection reagents (e.g., nucleic acid-based detection reagents, etc.) associated with the presence of dry eye disease in a subject.

A “biological sample” is a sample of biological material taken from a patient or subject. Biological samples include samples taken from bodily fluids, cells, and tissues (e.g., from a biopsy) or tissue preparations (e.g., tissue sections, homogenates, etc.). A “bodily fluid” is any fluid obtained or derived from a subject suitable for use in accordance with the invention. Such fluids include tears, saliva, and vitreous humor.

A “companion diagnostic” is a diagnostic test designed to identify subgroups of patients who may or may not benefit from a particular drug, who may have adverse reactions to the drug, or may require different dosages of the drug.

The term “drug rescue” refers to a drug or drug candidate in the context of the reevaluation of samples and/or data from discontinued clinical trials or pre-clinical development with new or improved evaluation methods.

The term “high-throughput” refers to the ability to rapidly process multiple specimens, for example, arrays or microarrays according to the invention, in an automated and/or massively parallel manner. On the other hand, the term “multiplex” refers to the concurrent performance of multiple experiments on a single device or in a single assay. For instance, a multiplex assay using an array according to the invention allows the simultaneous detection and/or measurement of a plurality of different biomarker species in a biological sample on a single device.

A “patentable” process, machine, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically excludes the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

A “plurality” means more than one.

The term “positive going” marker as that term is used herein refer to a marker that is determined to be elevated in subjects suffering from a disease or condition, relative to subjects not suffering from that disease or condition. The term “negative going” marker as that term is used herein refer to a marker that is determined to be reduced in subjects suffering from a disease or condition, relative to subjects not suffering from that disease or condition.

The term “sample profiling” refers to a representation of information relating to the characteristics of a biological sample, for example, tear fluid, recorded in a quantified way in order to determine patterns or signatures of biomolecules in the particular sample.

As used herein, the singular forms “a”, “an”, and “the” include plural references unless the context clearly dictates otherwise.

As used herein, the term “about” refers to approximately a +/−10% variation from the stated value. It is to be understood that such a variation is always included in any given value provided herein, whether or not it is specifically referred to.

### SUMMARY OF THE INVENTION

It is an object of the invention to provide articles, devices, kits, and methods for diagnosing underlying immunopathological mechanisms involved in or associated or correlated with autoimmune and inflammatory conditions or diseases, as well as articles, devices, kits, and methods for detection of biomarkers associated with immunopathological mechanisms involved in or associated or correlated with autoimmune and inflammatory conditions or diseases in biological samples, particularly samples from disease manifested organs or tissues, such as tear fluid, saliva, or vitreous humor, obtained from subjects. As described herein, simultaneous assessment of two or more naturally occurring biomarker species associated with the immunopathology and clinical manifestation in autoimmune and inflammatory conditions can be used for diagnosis of one or more underlying biological mechanisms, particularly underlying immunopathological mechanisms (e.g., to screen for an initial occurrence, recurrence, progression, etc.), different immunopathological mechanisms involved in or associated or correlated with immune mediated diseases (autoimmune and inflammatory diseases), disease stratification (that is, to identify subjects suffering from immune mediated disease based on underlying molecular mechanisms and/or pathways such that the subject's disease subtype thereof can be determined), diagnostic of the degree of disease severity, staging, monitoring (e.g., to assess whether a subject is experiencing deterioration or improvement of clinical status over time), prognosis (e.g., predicting a future medical outcome, such as improved or worsening disease, a decreased or increased morbidity risk, or responsiveness to a particular therapeutic regimen), categorizing and determination of further diagnosis and treatment regimens in subjects suffering or at risk of suffering from immune-mediated disease (e.g., dry eye disease) or recurrence thereof, as well as in the context of drug development.

Thus, in one aspect, the invention concerns diagnostic methods of underlying immunopathological mechanisms in autoimmune and inflammatory diseases. In general, these methods include contacting a biological or clinical sample (e.g., a tear sample) obtained from a subject known to have or suspected of having an autoimmune or inflammatory disease with a detection reagent that binds a biomarker associated with an underlying immunopathological mechanism in immune mediated diseases. The detection reagent can be used to determine if the biomarker associated with the disease mechanism is present in the sample in an amount indicative of underlying disease molecular mechanism. Preferred biomarkers include IL-1beta (IL-1b), IL-1alpha (IL-1a), IL-1 receptor antagonist (IL-1Ra), IL-15, IL-7, IL-2, IL-18, IL-8, IL-12p70, IL-12p40, IL-17, IL-23, CXCL-10, ICAM-1, MMP-9, MMP-9, MIP-1alpha (MIP-1a), MIP-1 beta (MIP-1b), Complement 3, alpha1-antitrypsin, apolipoprotein A1, apolipoprotein CIII, and IgM, and/or any of derivative or fragment of any of the foregoing. Detection reagents preferably comprise an antibody or antigen-binding antibody fragment. While monoclonal antibodies are preferred, polyclonal antibodies can also be utilized. In some embodiments, two or more different detection reagent species may be employed, in which event each detection reagent species preferably binds a different biomarker species. In some embodiments, however, two or more detection reagent species may target the same or different epitopes on the same biomarker.

In a related aspect, the present invention concerns methods for evaluating a biological sample from a subject to assess whether it contains one or more biomarker species associated with immunopathological mechanisms involved in or associated or correlated with autoimmune and inflammatory conditions or diseases. These methods comprise performing an assay (e.g., an immunoassay) configured to detect biomarker species in a biological sample, such as a tear fluid or saliva, obtained from a subject. The assay result, for example, a measured level of the targeted biomarker(s), is then correlated with the presence of a proinflammatory T cell-mediated, autoantibody-mediated, or innate immune-mediated pathological mechanism, and may be used for one or more of disease stratification, diagnosis, prognosis, staging, classifying, monitoring, and treatment.

Another aspect of the invention concerns diagnostic kits of for assessing or detecting an underlying immunopathological mechanism involved in one or more autoimmune and/or inflammatory diseases or conditions. Such kits typically include at least two detection reagent species that each independently bind a biomarker species associated with a pathological mechanism different from biomarker species bound by the other detection reagent species in the kit and instructions for using the detection reagent to analyze a biological sample obtained from a subject to determine if the biological sample contains an amount of the biomarkers indicative of one of the underlying immunopathological mechanisms. Kits that provide instructions for assessing one or more biomarker signatures (e.g., the levels (e.g., amounts, concentrations, etc.) of two or more biomarkers whose presence in a sample can be assayed using the reagents in the particular kit), for example, for classifying (or stratifying) which mechanistic subtype the subject may be afflicted with, if any.

Still another aspect of the invention relates to diagnostic methods wherein two or more detection reagent species are used to determine whether a biomarker signature indicative of an underlying immunopathological mechanism is present in a biological sample (e.g., a tear sample) obtained from a subject. Such methods may further comprise using a biomarker signature differentiating distinct underlying mechanisms to determine which biomarker signature is present, which information can then be used, for example, to classify which dry eye (or other immune-mediated) disease subtype afflicts the subject from whom the tear sample was obtained. Such information will have use in determining which therapeutic intervention(s) to use (e.g., which drug(s) to administer), as well as in the context of clinical trials, for example, to determine whether a patient should receive a therapeutic intervention based on a drug that modulates a specific biochemical pathway or biological response (e.g., inflammation) relevant in the context of an immune-mediated disease or sub-type thereof. Such methods can also be used to select patients for participation in an autoimmune or inflammatory disease clinical trial, especially those wherein a drug candidate is being studied for modulation of a specific biochemical pathway or biological response relevant in the context of an immune-mediated disease or sub-type thereof.

Several representative embodiments of this aspect are as follows. Under homeostatic conditions, proinflammatory type 1 and type 17 cell immunities (which are important for defense against invading pathogens) are balanced by type 2 cell immunity. In autoimmune or inflammatory diseases, this balance is altered in tissues or organs that are effected by immunopathological changes and clinical manifestations. For detection of a biomarker signature in patients, a higher ratio of Th17-associated cytokines (such as IL-17A) over Th2-associated cytokines (such as IL-10) than that in normal control subjects would indicate the presence of pathogenic Th17 immunity and proinflammatory type 17 cascades as an underlying immunopathological mechanism in these patients. These patients would likely respond to or benefit from a treatment that inhibits or dampens the type 17 immunity, such as an antagonist of IL-17, IL-23, IL-1, or IL-15 activity, or stimulates the anti-inflammatory aspect of the type 2 immunity, such as a drug that stimulates or enhances the production of IL-10, such as a treatment with IL-18. Patients who are determined to have a lower ratio of IL-1Ra over IL-1 than that in normal controls, indicating an active IL-1 pathway is involved in the pathophysiology of these patients, would likely respond t or benefit from IL-1 modulators, such as anti-IL-1 antibodies, IL-1 Ra, or blocker of IL-1 signaling. Patients who are found to have a biomarker signature of immunopathology mediated by active complement, IL-8/neutrophile would likely respond to or benefit from complement inhibitors, treatments that inhibit neutrophil infiltration, activation, or activity, B cell modulators, Toll-Like-Receptor inhibitors that block the induction of type 1 interferon, or an IL-18 inhibitor such as IL-18BP, anti-IL-18 antibodies, or another blocker of IL-18 signaling.

Another representative embodiment of this aspect is to collect biological samples, such as blood or body fluids, such as saliva or tears, from SS patients or subjects known to be or suspected of suffering from SS syndrome, analyze the biomarker signatures in the biological samples, and stratify patients into subtypes based on underlying molecular pathological mechanisms, for example, patients having a biomarker signature indicative of enhanced complement activity and microvascular leakage (such as higher level of complement and alpha 1 antitrypsin (ATT) than in normal controls); patients with pathogenic type 17 immunity being the primary pathological mechanism (such as higher ratio of IL-17 over IL-10 or higher ratio of IL-1 over IL-1 Ra than in normal controls); or enhanced neutrophil activity and suppressed T cell immunity (such as higher level of IL-8 and lower level of IL-23 or IL-1 or IL-15 or IL-12 than in normal controls). Each subtype of patients would be treated with a different drug to targeting the corresponding pathological mechanism(s), as is the case. Similarly with other immune mediated diseases.

In various related aspects, the present invention also relates to devices and kits for performing the methods described herein. Suitable kits comprise reagents sufficient for performing an assay according to the invention, together with instructions for performing the described threshold comparisons

Features and advantages of the invention will be apparent from the following detailed description, and appended claims.

### BRIEF DESCRIPTION OF THE DRAWINGS

A brief summary of each of the figures and tables described in this specification are provided below. This application contains at least one figure executed in color. Copies of this application with color drawings will be provided upon request and payment of the necessary fee.

FIG. 1 provides several panels of scatter plots showing correlations of tear protein markers in a group of non-dry eye control subjects: tear levels of IL-10, IFN-gamma, IL-2 and IL-17 with IL-18 (A), and with IL-1beta (B), respectively, Part A shows four panels of scatter plots in a group of non-dry eye control subjects: tear levels of IL-10, IFN-gamma, IL-2 and IL-17 with IL-18 (A), and with IL-1 beta (B), respectively. Similarly, Part B shows four panels of scatter plots in a group of non-dry eye control subjects: tear levels of IL-10, IFN-gamma, IL-2 and IL-17 with IL-18 (A), and with IL-1 beta (B), respectively. In each of Panels A and B, protein biomarker concentration is in Log 10 scale and each square represents one eye.

FIG. 2 provides four panels of scatter plots showing correlations of tear protein markers in a group of moderate-to-severe aqueous deficient dry eye patients: tear levels of IL-1beta vs. IL-17, IL-18 vs. IL-17, IL-18 vs. IL-10, and IL-23 vs. IL-17. Tear samples with higher IL-17 level are in brighter color. Protein biomarker concentration is in Log 10 scale and each square represents one eye.

FIG. 3 provides three Panels, A, B, and C, each showing four panels of scatter plots in a group of moderate-to-severe aqueous dry eye patients, showing multiple subgroups of patients: tear level of IL-18 vs. Log 10 (IL-17/IL-10), IL-1beta vs. Log 10 (IL-17/IL-10), MMP.3 vs. MMP.9 and Complement 3 vs. IL-8. Protein biomarker concentration is in Log 10 scale and each square represents one eye.

FIG. 4 provides several panels of scatter plot showing correlations between pairs of protein markers in dry eye patients in a clinical study.

FIG. 5 shows multivariate analysis of tear markers in dry eye patients. 2-way unsupervised hierarchical clustering of tear protein markers and patients. Column: patients, Row: tear protein markers. For each tear protein marker concentration, green=low, red=high. Group: dry eye patient groups with mild, moderate, and severe corneal staining at screening represented by yellow, orange and red. Site: two investigator sites represented by grey and red.

FIG. 6 has two panels, A and B, each showing shows principal component analysis (PCA). Panel A: PCA of dry eye patients using tear protein markers. Panel B: PCA of both control subjects and dry eye patients. PCA plot: green=G1, blue=G2, red=G3, orange=G4, grey=Control.

FIG. 7 provides several panels of box plots and individual patient dot plots of several of select biomarkers in each subset of dry eye patients.

FIG. 8 provides bar graphs providing group means and 95% confidence intervals of select protein biomarkers on Day 0 (black) and Day 7 (grey) in each subgroup of dry eye patients in a clinical study.

FIG. 9 provides several panels of box plots and individual patient dot plots of select markers in each subset of patients in a second clinical study.

Table 1 lists the Pearson correlation coefficients between tear levels of IL-1alpha, IL-1 beta and IL-18 with Th1, Th2 and Th17 associated cytokines in control non-dry eye subjects.

Table 2 lists the clinical features of dry eye patients at screening visit in a clinical study.

Table 3 lists the clinical features of dry eye patients at screening visit in 2nd clinical study.

### DETAILED DESCRIPTION

As those in the art will appreciate, the following detailed description describes certain preferred embodiments of the invention in detail, and is thus only representative and does not depict the actual scope of the invention. Before describing the present invention in detail, it is understood that the invention is not limited to the particular aspects and embodiments described, as these may vary. It is also to be understood that the terminology used herein is for the purpose of describing particular embodiments only, and is not intended to limit the scope of the invention defined by the appended claims.

More specifically, the present invention relates to articles, devices, kits, and methods for diagnosis, differential diagnosis, disease stratification, monitoring, classifying, and determination of treatment regimens in subjects suffering or at risk of suffering from an autoimmune or inflammatory disease through measurement of one or more biomarkers associated with the disease.

### Biomarker Changes in Disease

The cellular changes that mark the transition from a healthy to a diseased state are frequently, if not always, mediated by changes in the level or type of constituent biomarkers, including proteins, nucleic acids, carbohydrates, and lipids. These changes can result from several different mechanisms, including changes in the abundance or expression level of certain proteins, the rate of transcription of DNA to mRNA or the translation of mRNA to protein, mRNA stability, the rate of protein turnover, or other metabolic processes. One, some, or all of these and other mechanisms may be modulated, with the result being that the synthesis and/or stability of one or more biomarker species is increased or decreased in a manner that can be detected in an assay of a biological sample. With particular regard to proteins, there may also be changes in the primary sequence of a protein conferred by alterations in the corresponding gene sequences, due to single nucleotide polymorphisms (SNPs), alternate mRNA splicing, genomic rearrangements, or any of several other mechanisms for genetic variation. There may also be changes in the processing and post-translational modification of proteins. For example, a protein may be differentially glycosylated such that alternative glycoforms can be detected.

### Analyte Detection

The presence and/or amount of a target analyte, e.g., a biomarker associated with dry eye disease, can be detected or measured in biological samples, particularly tears, obtained from subjects by any suitable method, including obtaining a small tear volume directly from a subject's eye, as well as via biopsy, swab, washing, or other technique useful to collect a biological fluid or cell sample from a patient. Particularly preferred biological samples are tear samples, as tear fluid is usually a readily accessible solution that can be obtained by relatively non-invasive sampling techniques.

Biomarkers are generally detected using biomarker-reactive reagent species immobilized on a substrate such as a solid support. A biomarker detection reagent species is one specifically reactive with an epitope of a biomarker now known or later discovered to be associated with dry eye disease. Thus, a detection reagent species refers to a reagent that is specifically reactive with a particular epitope of a biomarker antigen. Preferred detection reagent species comprise polyclonal, and even more preferably, monoclonal antibodies, or the antigen-binding fragments of such antibodies. A detection reagent may also include one or more other moieties, for example, a detectable label.

In this invention, one or more detection reagent species are immobilized on a suitable substrate, for example, plastic beads, on the surface of the detection zone of a lateral flow device, etc. In this way, the detection reagent(s) can be brought into contact with a small biological sample (e.g., from about 1 nanoliter (nL) to about 500 microliters (uL) of tear fluid) to determine if it contains one or more biomarkers associated with one immunopathological mechanism or another immunopathological mechanism in autoimmune and inflammatory diseases.

A biomarker detection array (or other configuration of multiple detection reagent species immobilized on one or more substrates) of the invention can also include other moieties reactive with biomolecules in a biological sample. For example, detection reagents reactive with disease-associated metabolites, proteins, and/or nucleic acids that encode them, can also be included. Detection reagents for these and/or other molecular mechanism-associated biomarkers can also be included in a panel or on an array according to the invention.

In preferred embodiments, the arrays of the invention comprise at least two detection reagent species, each of which corresponds to a specific biomarker.

As those in the art will appreciate, immunoassay formats are particularly preferred for implementing the instant invention. Immunoassays can provide qualitative, semi-quantitative, or quantitative output. Immunoassays are biochemical tests that measure the presence and/or level of one or more substances, i.e., analytes (e.g., biomarkers such as proteins, nucleic acids, etc.), in a biological sample, for example, a small volume of tear fluid, using the reaction of an antibody or antibodies to its antigen. The assay takes advantage of the specific binding of an antibody to its antigen to form an antibody-antigen complex, a representative example of a detection reagent-biomarker complex. Antigens or antibodies can be detected or measured. In the context of the invention it is generally biomarker species that are detected.

Numerous immunoassay formats are known to those of skill in the art, who understand that the signals obtained from an immunoassay are a direct result of complexes formed between one or more antibodies (a preferred detection reagent component) and polypeptides (a representative class of biomarker) containing the necessary epitope(s) to which the antibodies bind. As used herein, the term “relating a signal to the presence or amount” of an analyte reflects this understanding. As already described, assay signals are typically related to the presence or amount of an analyte through the use of a standard curve calculated using known concentrations of the analyte of interest. As the term is used herein, an assay is “configured to detect” an analyte if an assay can generate a detectable signal indicative of the presence or amount of a physiologically relevant concentration of the analyte.

In general, immunoassays involve contacting a sample containing or suspected of containing a biomarker of interest with at least one antibody (or antigen-binding antibody fragment) that specifically binds to the biomarker. A signal is then generated indicative of the presence or amount of complexes formed by the binding of polypeptides in the sample to the antibody. The signal is then related to the presence or amount of the biomarker in the sample. Numerous methods and devices are well known to the skilled artisan for the detection and analysis of biomarkers. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, and The Immunoassay Handbook, David Wild, ed., Elsevier 2005, each of which is hereby incorporated by reference in its entirety, including all tables, figures, and claims.

The assay devices and methods known in the art can utilize labeled molecules in various sandwich, competitive, or non-competitive immunoassay formats to generate a signal that is related to the presence or amount of the biomarker of interest. Other suitable assay formats also include chromatographic, mass spectrographic, and protein “blotting” methods. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. One skilled in the art also recognizes that robotic instrumentation, including but not limited to, Beckman ACCESS®, Abbott AXSYM®, Roche ELECSYS®, Dade Behring STRATUS® systems, are among the immunoassay analyzers that are capable of performing immunoassays. But any suitable immunoassay may be utilized, for example, enzyme-linked immunoassays (ELISA), radioimmunoassays (RIAs), competitive binding assays, and the like.

Antibodies or other polypeptides may be immobilized onto a variety of solid supports for use in assays. Solid phases that may be used to immobilize specific binding members include those developed and/or used as solid phases in solid phase binding assays. Examples of suitable solid phases include membrane filters, cellulose-based papers, beads (including polymeric, latex and paramagnetic particles), glass, silicon wafers, microparticles, nanoparticles, TentaGels, AgroGels, PEGA gels, SPOCC gels, and multiple-well plates. Antibodies or other detection reagents may be bound to specific zones of assay devices either by conjugating directly to an assay device surface, or by indirect binding. In an example of the later case, antibodies or other polypeptides may be immobilized on particles or other solid supports, and that solid support immobilized to the device surface.

Biological assays require methods for detection, and one of the most common methods for quantitation of results is to conjugate a detectable label to a protein or nucleic acid that has affinity for one of the components in the biological system being studied. Detectable labels may include molecules that are themselves detectable (e.g., fluorescent moieties, electrochemical labels, ecl (electrochemical luminescence) labels, metal chelates, colloidal metal particles, radioactive labels, etc.), as well as molecules that may be indirectly detected by production of a detectable reaction product (e.g., enzymes such as horseradish peroxidase, alkaline phosphatase, etc.) or through the use of a specific binding molecule which itself may be detectable (e.g., a labeled antibody that binds to the second antibody, biotin, digoxigenin, maltose, oligohistidine, 2,4-dintrobenzene, phenylarsenate, ssDNA, dsDNA, etc.).

Generation of a signal from the signal development element can be performed using various optical, acoustical, and electrochemical methods well known in the art. Examples of detection modes include fluorescence, radiochemical detection, reflectance, absorbance, amperometry, conductance, impedance, interferometry, ellipsometry, etc. In certain of these methods, the solid phase antibody is coupled to a transducer (e.g., a diffraction grating, electrochemical sensor, etc) for generation of a signal, while in others, a signal is generated by a transducer that is spatially separate from the solid phase antibody (e.g., a fluorometer that employs an excitation light source and an optical detector). This list is not meant to be limiting. Antibody-based biosensors may also be employed to determine the presence or amount of analytes that optionally eliminate the need for a labeled molecule.

Preparation of solid phases and detectable label conjugates often comprise the use of chemical cross-linkers. Cross-linking reagents contain at least two reactive groups, and are divided generally into homofunctional cross-linkers (containing identical reactive groups) and heterofunctional cross-linkers (containing non-identical reactive groups). Homobifunctional cross-linkers that couple through amines, sulfhydryls or react non-specifically are available from many commercial sources. Maleimides, alkyl and aryl halides, alpha-haloacyls and pyridyl disulfides are thiol reactive groups. Maleimides, alkyl and aryl halides, and alpha-haloacyls react with sulfhydryls to form thiol ether bonds, while pyridyl disulfides react with sulfhydryls to produce mixed disulfides. The pyridyl disulfide product is cleavable. Imidoesters are also very useful for protein-protein cross-links. A variety of heterobifunctional cross-linkers, each combining different attributes for successful conjugation, are commercially available

To obtain quantitative or semi-quantitative results, results must be compared to standards of a known concentration. This is usually done though the use of one or more standard curves. The position of the curve at response of the unknown is then examined, and so the quantity of the unknown found.

Detecting the quantity of a particular protein or other biomarker species can be achieved by a variety of methods, any of which can be readily adapted for practice of the invention. ELISA is a commonly used technique for detecting antibody or antigen levels. One of the most common methods is to label either the antigen or antibody with an enzyme, radioisotope, or fluorescence. Other suitable techniques include agglutination, flow cytometry, Luminex assays, cytometric bead arrays, and lateral flow, among others now know or later developed.

Immunoassays can involve “sandwich” approaches in which the analyte to be detected (e.g., a protein found in tears that is associated with dry eye disease) is bound by two other entities, for example, by a capture reagent immobilized on a substrate and specific for the target biomarker species and a labeled detection reagent that binds to another epitope on the targeted biomarker species. In this way the “sandwich” can be used to measure the amount of the biomarker bound between the capture and detection reagents. Sandwich assays are especially valuable to detect analytes present at low concentrations or in complex solutions (e.g., tears) containing high concentrations of other molecular species. As is known, in these sorts of assays a “capture” reagent is immobilized on a solid phase (i.e., on a substrate) such as a glass slide, plastic strip, or microparticle. A liquid biological sample (e.g., a tear sample) known or suspected to contain the targeted biomarker is then added and allowed to complex with the immobilized capture reagent. Unbound products are removed and the detection reagent is then added and allowed to bind to biomarker species that has been “captured” on the substrate by the capture reagent, thus completing the “sandwich”. These interactions can then be used to quantitate the amount of the captured biomarker species present in the biological sample.

As will be appreciated, a plurality of different dry eye disease-associated capture reagent species (e.g., 2, 5, 10, 25, 50, 100, or more capture reagent species) can be immobilized on the substrate (or on different substrates, for example, different distinguishable microparticles) in order to detect, via “capture”, a plurality of different biomarker species in a single multiplex assay. To allow simultaneous detection of multiple biomarker species in a single assay, a multiplex assay format can be used. Multiplex formats provide an array of different moieties that allow simultaneous detection of multiple analytes (e.g., different biomarker species) at multiple array addresses on a single substrate. Alternatively, when a panel of the invention is spread across multiple substrates, for example, in embodiments where different dry eye disease-associated capture or detection reagent species are immobilized on substrates that can be distinguished (e.g., differentially labeled microparticles configured for use in Luminex assays), multiple array addresses can still be readily distinguished.

Thus, in certain embodiments, the assay methods of the invention utilize immunoassays. In certain embodiments, reagents for performing such assays are provided in an assay device, and such assay devices may be included in such a kit. Preferred reagents can comprise two or more independently selected solid phase detection reagents, each of which comprises an antigen reagent species specific for its target biomarker, immobilized on the same or different substrate (here, any suitable solid support). In the case of sandwich immunoassays, such reagents can also include one or more detectably labeled antibodies, the detectably labeled antibody comprising antibody that detects the intended biomarker target(s) bound to a detectable label. Additional optional elements that may be provided as part of an assay device are described hereinafter. Numerous methods and devices are well known to the skilled artisan for the detection and analysis of biomarkers. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, and The Immunoassay Handbook, David Wild, ed. Stockton Press, New York, 1994.

Preparation of substrates, solid phases, and detectable label conjugates often comprise the use of chemical cross-linkers. Cross-linking reagents contain at least two reactive groups, and are divided generally into homofunctional cross-linkers (containing identical reactive groups) and heterofunctional cross-linkers (containing non-identical reactive groups). Homobifunctional cross-linkers that couple through amines, sulfhydryls or react non-specifically are available from many commercial sources. Maleimides, alkyl and aryl halides, alpha-haloacyls and pyridyl disulfides are thiol reactive groups. Maleimides, alkyl and aryl halides, and alpha-haloacyls react with sulfhydryls to form thiol ether bonds, while pyridyl disulfides react with sulfhydryls to produce mixed disulfides. The pyridyl disulfide product is cleavable. Imidoesters are also very useful for protein-protein cross-links. A variety of heterobifunctional cross-linkers, each combining different attributes for successful conjugation, are commercially available.

Certain aspects of the present invention concern kits. Such kits comprise biomarker detection panels according the invention in order to allow performance of the methods of the invention. As such, such kits can also include devices and instructions for performing one or more of the methods described herein. The instructions can be in the form of labeling, which refers to any written or recorded material that is attached to, or otherwise accompanies a kit at any time during its manufacture, transport, sale, or use. For example, the term labeling encompasses advertising leaflets and brochures, packaging materials, instructions, computer storage media, as well as writing imprinted directly on kits.

In preferred embodiments a panel of the invention will also include controls, preferably at least one positive and one negative at least one positive control. Any suitable set of controls can be selected.

Additional clinical indicia may be combined with the biomarker assay result(s) of the present invention. These include other biomarkers associated or correlated with dry eye disease. Other clinical indicia which may also be combined with the assay result(s) of the present invention includes patient demographic information (e.g., weight, sex, age, race, smoking status), medical history (e.g., family history, type of surgery, pre-existing or previous diseases), and genetic information. Combining assay results/clinical indicia in this manner can comprise the use of multivariate logistical regression, loglinear modeling, neural network analysis, n-of-m analysis, decision tree analysis, etc. This list is not meant to be limiting.

The term “diagnosis” as used herein refers to methods by which the skilled artisan can estimate and/or determine the probability (“a likelihood”) of whether or not a patient is suffering from a given disease or condition. In the case of the present invention, “diagnosis” includes using the results of an assay, most preferably an immunoassay, of the present invention, optionally together with other clinical characteristics, to arrive at a diagnosis (that is, the occurrence or nonoccurrence) of disease underlying mechanism for the subject from which a sample was obtained and assayed. That such a diagnosis is “determined” is not meant to imply that the diagnosis is 100% accurate. Many biomarkers are indicative of multiple conditions. The skilled clinician does not use biomarker results in an informational vacuum, but rather test results are used together with other clinical indicia to arrive at a diagnosis. Thus, a measured biomarker level on one side of a predetermined diagnostic threshold indicates a greater likelihood of the occurrence of disease in the subject relative to a measured level on the other side of the predetermined diagnostic threshold.

Similarly, a prognostic risk signals a probability (“a likelihood”) that a given course or outcome will occur. A level or a change in level of a prognostic indicator, which in turn is associated with an increased probability of morbidity (e.g., worsening of the particular disease or condition) is referred to as being “indicative of an increased likelihood” of an adverse outcome in a subject.

In preferred diagnostic embodiments, the methods of the invention allow for diagnosing the occurrence or nonoccurrence of a disease, particularly dry eye disease, and the assay result(s) is/are correlated to the occurrence or nonoccurrence of the particular disease. For example, each of the measured biomarker levels (e.g., as concentration(s)) may be compared to a threshold value, which may be different for each biomarker species (or other analyte or biomarker to be studied in a given assay). The terms “correlating”, “correlated with”, and “associated with” as used herein in reference to the use of biomarkers refers to comparing the presence or amount of the biomarker(s) in a patient to its presence or amount in persons known to suffer from, or known to be at risk of, a given condition; or in persons known to be free of a given condition. Often, this takes the form of comparing an assay result in the form of a biomarker concentration to a predetermined threshold selected to be indicative of the occurrence or nonoccurrence of a disease or the likelihood of some future outcome.

In this context, “diseased” is meant to refer to a population having one characteristic (the presence of a disease or condition or the occurrence of some outcome) and “nondiseased” is meant to refer to a population lacking the characteristic. While a single decision threshold is the simplest application of such a method, multiple decision thresholds may be used. For example, below a first threshold, the absence of disease may be assigned with relatively high confidence, and above a second threshold the presence of disease may also be assigned with relatively high confidence. Between the two thresholds may be considered indeterminate. This is meant to be exemplary in nature only.

Selecting a diagnostic threshold involves, among other things, consideration of the probability of disease, distribution of true and false diagnoses at different test thresholds, and estimates of the consequences of treatment (or a failure to treat) based on the diagnosis. For example, when considering administering a specific therapy that is highly efficacious and has a low level of risk, few tests are needed because clinicians and patients are willing to accept substantial diagnostic uncertainty. On the other hand, in situations where treatment options are less effective and more risky, clinicians and patients often require a higher degree of diagnostic certainty before adopting a particular treatment regimen. Thus, cost/benefit analysis is involved in selecting a diagnostic threshold.

A variety of methods may be used by to arrive at a desired threshold value for use in these methods. For example, the threshold value may be determined from a population of normal subjects by selecting a concentration representing the 75^{th}, 85^{th}, 90^{th}, 95^{th}, or 99^{th} percentile of the biomarker measured in such normal subjects. Alternatively, the threshold value may be determined from a “diseased” population of subjects, e.g., those suffering from a disease such as a dry eye disease or having a predisposition for dry eye disease, its recurrence, or progression, by selecting a concentration representing the 75^{th}, 85^{th}, 90^{th}, 95^{th}, or 99^{th} percentile of the biomarker measured in such subjects. In another alternative, the threshold value may be determined from a prior measurement of the biomarker in the same subject, where a prior “baseline” result is used to monitor for temporal changes in a biomarker level; that is, a temporal change in the level of the biomarker in the subject may be used for diagnostic and/or prognostic purposes.

The foregoing discussion is not meant to imply, however, that the levels of biomarkers measured in assays of the invention must be compared to corresponding individual thresholds. Methods for combining assay results can comprise the use of multivariate logistical regression, loglinear modeling, neural network analysis, n-of-m analysis, decision tree analysis, calculating ratios of markers, etc. This list is not meant to be limiting. In these methods, a composite result that is determined by combining individual biomarker data or results may be treated as if it is itself a marker; that is, a threshold may be determined for the composite result as described herein for individual biomarkers, and the composite result for an individual patient compared to this threshold.

Population studies may also be used to select a decision threshold. Receiver Operating Characteristic (“ROC”) arose from the field of signal detection theory developed during World War II for the analysis of radar images, and ROC analysis is often used to select a threshold able to best distinguish a “diseased” subpopulation from a “nondiseased” subpopulation. A false positive in this case occurs when a subject tests positive, but actually does not have the disease. A false negative, on the other hand, occurs when the person tests negative, suggesting they are healthy, when they actually do have the disease. To draw a ROC curve, the true positive rate (TPR) and false positive rate (FPR) are determined as the decision threshold is varied continuously. Since TPR is equivalent with sensitivity and FPR is equal to 1-specificity, the ROC graph is sometimes called the sensitivity versus (1-specificity) plot. A perfect test will have an area under the ROC curve of 1.0; a random test will have an area of 0.5. A threshold is selected to provide an acceptable level of specificity and sensitivity.

Thus, the ability of a particular test to distinguish two populations can be established using ROC analysis. For example, ROC curves established from a “first” subpopulation which is predisposed to future disease or disease-related changes, and a “second” subpopulation which is not so predisposed can be used to calculate a ROC curve, and the area under the curve provides a measure of the quality of the test. Preferably, the tests described herein provide a ROC curve area greater than 0.5, preferably at least 0.6, more preferably 0.7, still more preferably at least 0.8, even more preferably at least 0.9, and most preferably at least 0.95.

In certain aspects, the measured concentration of one or more target biomarkers (e.g., disease-associated serum autoantibodies), or a composite of results, may be treated as continuous variables. For example, any particular concentration can be converted into a corresponding probability of some outcome for the subject. In yet another alternative, a threshold that can provide an acceptable level of specificity and sensitivity in separating a population of subjects into “bins” such as a “first” subpopulation (e.g., which is predisposed to one or more future changes in disease status, the occurrence or recurrence of disease, a disease classification or stratification, etc.) and a “second” subpopulation which is not so predisposed.

As discussed above, suitable tests may exhibit one or more of the following results on these various measures: a specificity of greater than 0.5, preferably at least 0.6, more preferably at least 0.7, still more preferably at least 0.8, even more preferably at least 0.9 and most preferably at least 0.95, with a corresponding sensitivity greater than 0.2, preferably greater than 0.3, more preferably greater than 0.4, still more preferably at least 0.5, even more preferably 0.6, yet more preferably greater than 0.7, still more preferably greater than 0.8, more preferably greater than 0.9, and most preferably greater than 0.95; a sensitivity of greater than 0.5, preferably at least 0.6, more preferably at least 0.7, still more preferably at least 0.8, even more preferably at least 0.9 and most preferably at least 0.95, with a corresponding specificity greater than 0.2, preferably greater than 0.3, more preferably greater than 0.4, still more preferably at least 0.5, even more preferably 0.6, yet more preferably greater than 0.7, still more preferably greater than 0.8, more preferably greater than 0.9, and most preferably greater than 0.95; at least 75% sensitivity, combined with at least 75% specificity; a ROC curve area of greater than 0.5, preferably at least 0.6, more preferably 0.7, still more preferably at least 0.8, even more preferably at least 0.9, and most preferably at least 0.95; an odds ratio different from 1, preferably at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less; a positive likelihood ratio (calculated as sensitivity/(1-specificity)) of greater than 1, at least 2, more preferably at least 3, still more preferably at least 5, and most preferably at least 10; and or a negative likelihood ratio (calculated as (1-sensitivity)/specificity) of less than 1, less than or equal to 0.5, more preferably less than or equal to 0.3, and most preferably less than or equal to 0.1.

In addition to threshold comparisons, other methods for correlating assay results to a patient classification (occurrence or nonoccurrence of disease, likelihood of an outcome, etc.) include decision trees, rule sets, Bayesian methods, and neural network methods. These methods can produce probability values representing the degree to which a subject belongs to one classification out of a plurality of classifications.

Measures of test accuracy may be obtained as described in Fischer et al., Intensive Care Med. 29: 1043-51, 2003, and used to determine the effectiveness of a given biomarker. These measures include sensitivity and specificity, predictive values, likelihood ratios, diagnostic odds ratios, and ROC curve areas. The area under the curve (“AUC”) of a ROC plot is equal to the probability that a classifier will rank a randomly chosen positive instance higher than a randomly chosen negative one. The area under the ROC curve may be thought of as equivalent to the Mann-Whitney U test, which tests for the median difference between scores obtained in the two groups considered if the groups are of continuous data, or to the Wilcoxon test of ranks.

### Antibodies

Antibodies (or antigen-binding antibody fragments and the like) used in the immunoassays described herein preferably specifically bind to a biomarker of the present invention. The term “specifically binds” is not intended to indicate that an antibody binds exclusively to its intended target since an antibody is capable of binding to any molecule displaying the epitope(s) to which the antibody binds. Rather, an antibody “specifically binds” if its affinity for its intended target is about 5-fold greater when compared to its affinity for a non-target molecule which does not display the appropriate epitope(s). Preferably the affinity of the antibody will be at least about 5-fold, preferably 10-fold, more preferably 25-fold, even more preferably 50-fold, and most preferably 100-fold or more, greater for a target molecule than its affinity for a non-target molecule. In preferred embodiments, preferred antibodies bind with affinities of at least about 10⁶ M⁻¹ or 10⁷ M⁻¹ to about 10¹² M⁻¹ and preferably between about 10⁸ M⁻¹ to about 10⁹ M⁻¹, about 10⁹ M⁻¹ to about 10¹⁰ M⁻¹, or about 10¹⁰ M⁻¹ to about 10¹² M⁻¹.

Affinity is calculated as K_{d}=k_{off}/kₒₙ (k_{off} is the dissociation rate constant, Kₒₙ is the association rate constant and K_{d} is the equilibrium constant). Affinity can be determined at equilibrium by measuring the fraction bound (r) of labeled ligand at various concentrations (c). The data are graphed using the Scatchard equation: r/c=K(n−r): where r=moles of bound ligand/mole of receptor at equilibrium; c=free ligand concentration at equilibrium; K=equilibrium association constant; and n=number of ligand binding sites per receptor molecule. By graphical analysis, r/c is plotted on the Y-axis versus r on the X-axis, thus producing a Scatchard plot. Antibody affinity measurement by Scatchard analysis is well known in the art.

Numerous publications discuss the use of phage display technology to produce and screen libraries of polypeptides for binding to a selected analyte. See, e.g, U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome that encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target analyte can then be synthesized in bulk by conventional means. See, e.g., U.S. Pat. No. 6,057,098.

The antibodies that are generated by these methods may then be selected by first screening for affinity and specificity with the purified biomarker of interest and, if required, comparing the results to the affinity and specificity of the antibodies with biomarkers that are desired to be excluded from binding. The screening procedure can involve immobilization of the purified biomarkers in separate wells of microtiter plates. The solution containing a potential antibody or groups of antibodies is then placed into the respective microtiter wells and incubated for about 30 min to 2 h. The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) are present.

The antibodies so identified may then be further analyzed for affinity and specificity in the assay design selected. In the development of immunoassays for a target protein or other type of biomarker, the purified target analyte acts as a standard with which to judge the sensitivity and specificity of the immunoassay using the antibodies that have been selected. Because the binding affinity of various antibodies may differ, and since certain antibody pairs (e.g., in sandwich assays) may interfere with one another sterically, etc., assay performance of an antibody may be a more important measure than absolute affinity and specificity of an antibody.

### Applications

The detection reagents, panels, arrays, and kits of the invention have numerous applications, including to monitor, prognose, diagnose, or in conjunction with treatment of a subject or patient having an autoimmune or inflammatory disease, for example, dry eye disease.

The arrays of the invention can be used to assess biological samples from patients known to have, suspected of having, or to have been previously diagnosed and/or treated for having, a particular disease, for example, a dry eye disease such as Sjogren's Syndrome, as well as to screen subjects not previously known or suspected to have a particular disease. At the time of screening, the subject or patient may be symptomatic or asymptomatic. Biomarker levels corresponding to some or all of the biomarker-reactive reagent species, or antigens, disposed on the array can be used prognostically, for example, to determine if a patient's disease is amenable to a particular treatment, to monitor disease progression and/or effectiveness of a therapeutic regimen, to assess disease aggressiveness of disease, and/or to identify likelihood of recurrence. The arrays of the invention can also be employed for diagnostic and screening purposes. For example, arrays can be configured to use in diagnosing one or more dry eye diseases.

The devices and arrays of the invention can also be used as a companion diagnostic, for example, to identify patients as likely responders or non-responders to a particular drug treatment or other therapeutic regimen, as well as for assessing the stage of a patient's disease as biomarker profiles are likely to change during disease progression. For example, tumors express different proteins (and thus produce different antigens) to meet the different requirements at each phase of development. Similarly, autoimmune diseases can “flare” at different times.

Data sets from diseased samples can also be correlated with clinical data. Antibody profiles can be used to predict disease severity or clinical outcome, which will be useful for prognostic applications. The use of biomarker panels will allow different stages of disease to be assessed, as the biomarker profile of a given sample will allow the particular stage of a given disease to be discerned, thereby allowing the most effective therapeutic intervention(s) to be employed.

The devices and arrays of the invention will also find use in drug development, both in the discovery and clinical development phases, particularly for biologic drugs such as antibodies and other recombinant proteins as well as cell- or vesicle-based drug delivery systems. Drugs of this class can, at least in some cases, elicit immune responses that can be advantageous (e.g., positive response to a vaccine) or harmful (e.g., severe adverse autoimmune reaction). Similarly, immune responses can also result from the administration of small molecule drugs, as a result of changes to cells and tissues following administration of the drug. The ability to monitor immune responses to biologic and small molecule drugs in clinical trials has never been more important. There is value in monitoring not only cellular immune responses but also humoral immune responses, and comparison of serum antibody profiles before and after treatment can help predict a favorable drug response. Positive responders to a drug will exhibit a different baseline humoral immune status to their disease. This is especially valuable in the case of immunomodulator class drugs that work by modifying an existing immune response rather than stimulating one de novo. By comparing data sets from non-responders to those who respond positively or negatively to a particular drug (or drug combination), panels can be defined for analyzing different groups of autoantibodies. Such panels will allow the identification of patients likely to respond to a particular therapy. Similarly, differences between responders and non-responders in the response profiles for a particular biomarker can be used to assess whether a patient is benefiting from a particular therapeutic regimen.

As will be appreciated, different clinical study designs will allow the development of biomarker panels that address different needs within drug development and therapy. For example, identifying responders versus non-responders will allow clinicians to select responders prior to treatment through the use of a companion diagnostic test based on response-predictive biomarker panel profile. Similarly, to select patient cohorts in clinical trials, biomarker profiles predictive for a positive drug response can be used to screen subjects prior to their recruitment into a clinical trial. This will ensure that only suitable candidates are included, and it may also be useful in gaining early drug approval. Also, information on drug non-response can assist regulatory bodies during consideration of drugs for approval or during post-approval surveillance (i.e., during a Phase IV clinical trial).

Another area of drug development where the instant invention will find application is in the area of “drug rescue” by helping to define the patient population(s) amenable to successful treatment as well as those who are unlikely to respond, or perhaps even more important, those who will experience an adverse reaction if administered the drug. In other words, a retrospective analysis of patient samples from a drug candidate that failed at some point in clinical development can be used to define the biomarker panel profile(s) (or signature(s)) predictive of a positive drug response. That information can then be used to define subsequent patient cohorts for further study and treatment. This process, which may be iterated, can revitalize drugs that have fallen out of conventional clinical development due to poor or insufficient evidence of efficacy. The biomarker panel profile(s) predictive of a positive drug response can then be used to reselect likely responders, which can lead to further clinical evaluation of the previously failed drug candidate but with a much greater likelihood of ultimately achieving drug approval.

### EXAMPLES

The following Examples are provided to illustrate certain aspects of the present invention and to aid those of skill in the art in its practice. These Examples are in no way to be considered to limit the scope of the invention in any manner, and those having ordinary or greater skill in the applicable arts will readily appreciate that the specification thoroughly describes the invention and can be readily applied to carry out the objects and obtain the ends and advantages mentioned, as well as those inherent therein.

### Example 1

### Distinct Underlying Immunopathological Mechanisms in Dry Eye Disease

### 1. Introduction

This example describes a study that involved the identification and characterization of distinct underlying inflammatory cascades involved in the pathophysiology and clinical manifestation in an autoimmune or inflammatory condition, such as dry eye. In this study, patients with dry eye and normal control subjects were analyzed at molecular level by profiling protein biomarker in tears. Methods were developed for identifying molecular components and underlying immunopathological mechanisms that are involved in resulting in patient heterogeneity at molecular mechanistic level in autoimmune and inflammatory conditions. Distinct underlying mechanisms and associated biomarker signature were identified.

### 2. Purpose

In this study, the objectives were identification of robust biomarkers and biomarker signature of underlying mechanisms, disease activity, classifying patient subgroups (either subtypes or different disease stages or status), development of biomarker signatures for patient classification, stratification and treatment, and selection of specific therapeutic targets and pathways.

### 3. Materials and Methods

Tears were collected from twenty-two asymptomatic control subjects and 59 aqueous deficient DED patients, for a systematic analysis of 43 protein biomarkers. These biomarkers are known to be immune mediators or to otherwise have potential immunopathological relevance. The analysis utilized a microbead-based multiplex immunoassay for each of these biomarkers. A different antibody-based detection reagent was used for each marker.

### 4. Results

### 1) Strong Positive Correlation Between Levels of IL-18 and Th2-Associated Cytokines and Between IL-18 and Th1-Associated Cytokines Under Homeostatic Condition

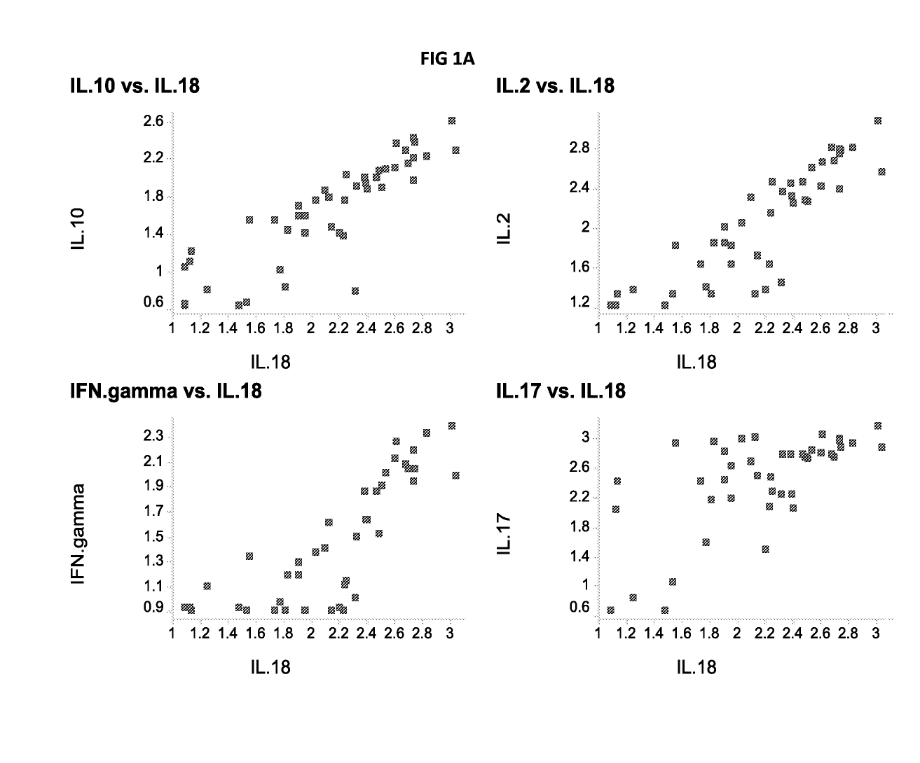

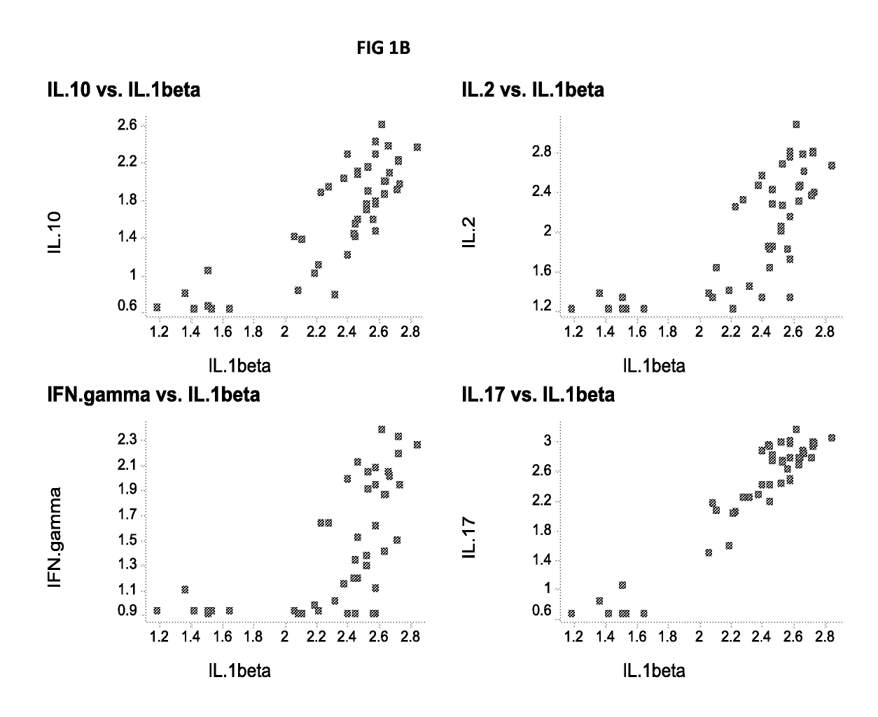

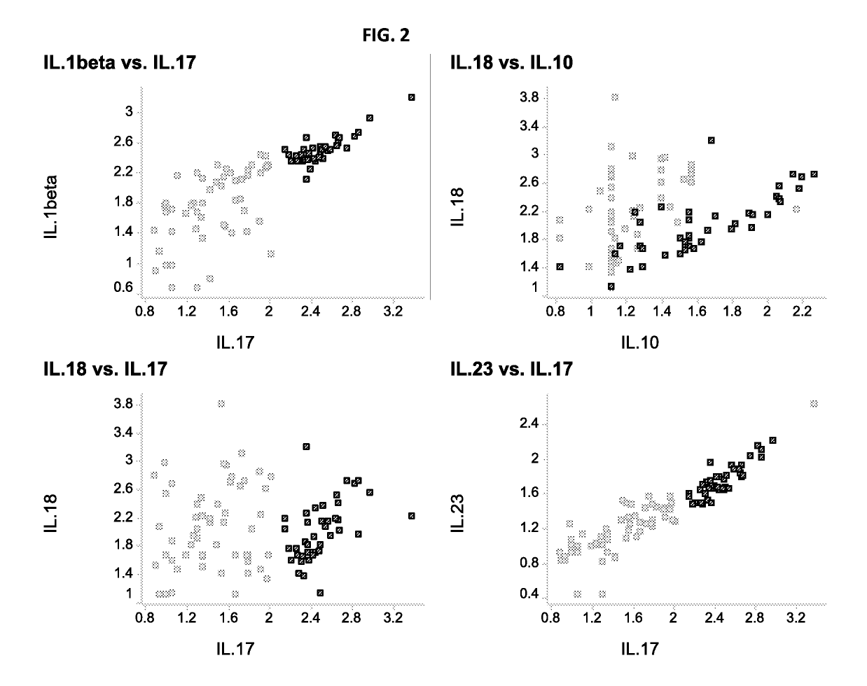

In control non-dry eye subjects, tear levels of IL-18 were strongly positively correlated with those of IL-10, IL-4, IL-5, IL-3 and GM-CSF; IL-18 levels also strongly correlated with IFN-g, IL-2 and TNF-a, but less so with IL-17; IL-1b (and IL-1a to less extent) correlated strongly with IL-17; IL-23 also strongly correlated with IL-17. Furthermore, IL-1 and IL-18 correlated with each other, and Th2 cytokines also correlated strongly with each other (r>0.90), with Th1 cytokines (r>0.85) and IL-17 (r: 0.71 to 0.83) as well. See Table 1 and FIG. 1. These results provide evidence for the close association between IL-18 activity and type2- and type1-associated cytokine production under homeostatic condition. Thus, IL-18 augments type1 and type2 responses, while IL-1 supports the IL17 response. The good correlations between IL-18 and IL-1, between type 2 and type 1 T cell cytokines, and between type 2 and type 17 cytokines ensure good balances under homeostatic condition between proinflammatory type 1 and type 17 cell immunities and the type 2 cell immunity.

TABLE 1
Pearson correlation coefficients between tear levels of IL-1alpha,
IL-1beta and IL-18 with Th1, Th2 and Th17 associated cytokines.

| Pearson Correlation Coefficient | IL-18 | IL-1alpha | IL-1beta |
|---|---|---|---|
| IFNgamma | 0.821 | 0.565 | 0.609 |
| IL-2 | 0.878 | 0.664 | 0.726 |
| TNFalpha | 0.856 | 0.596 | 0.636 |
| IL-10 | 0.866 | 0.745 | 0.819 |
| GM-CSF | 0.841 | 0.717 | 0.810 |
| IL-3 | 0.829 | 0.692 | 0.780 |
| IL-4 | 0.843 | 0.677 | 0.773 |
| IL-5 | 0.879 | 0.617 | 0.683 |
| IL-17 | 0.733 | 0.854 | 0.952 |
| IL-18 | 1.000 | 0.709 | 0.736 |
| IL-1alpha | 0.709 | 1.000 | 0.855 |
| IL-1beta | 0.736 | 0.855 | 1.000 |

### 2) Under Autoimmune or Inflammatory Condition, the Correlations and Balances Between IL-18 and IL-1, and Between Type 2 and Type 17 Cell Immunities were Altered

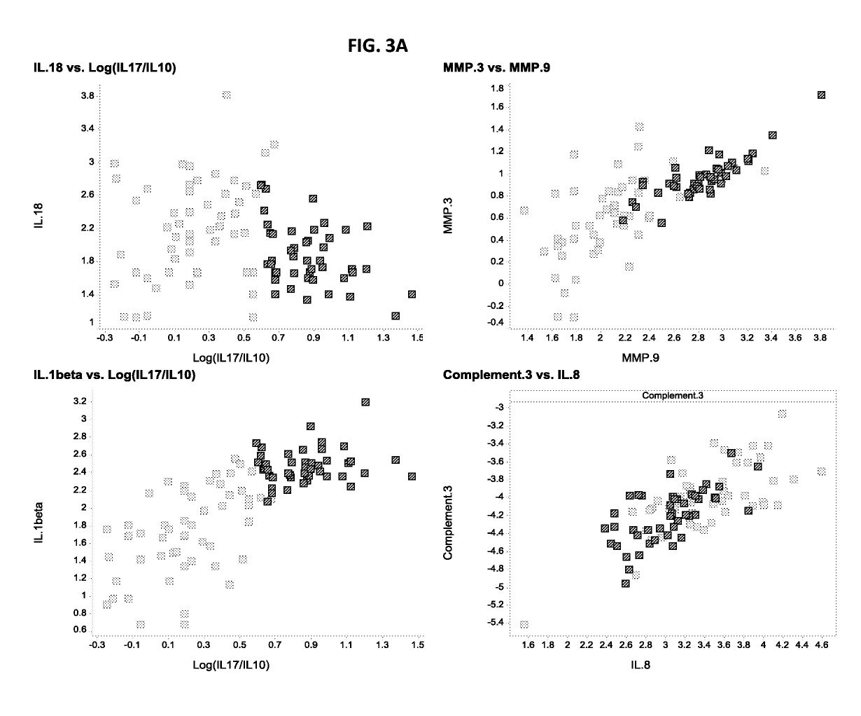

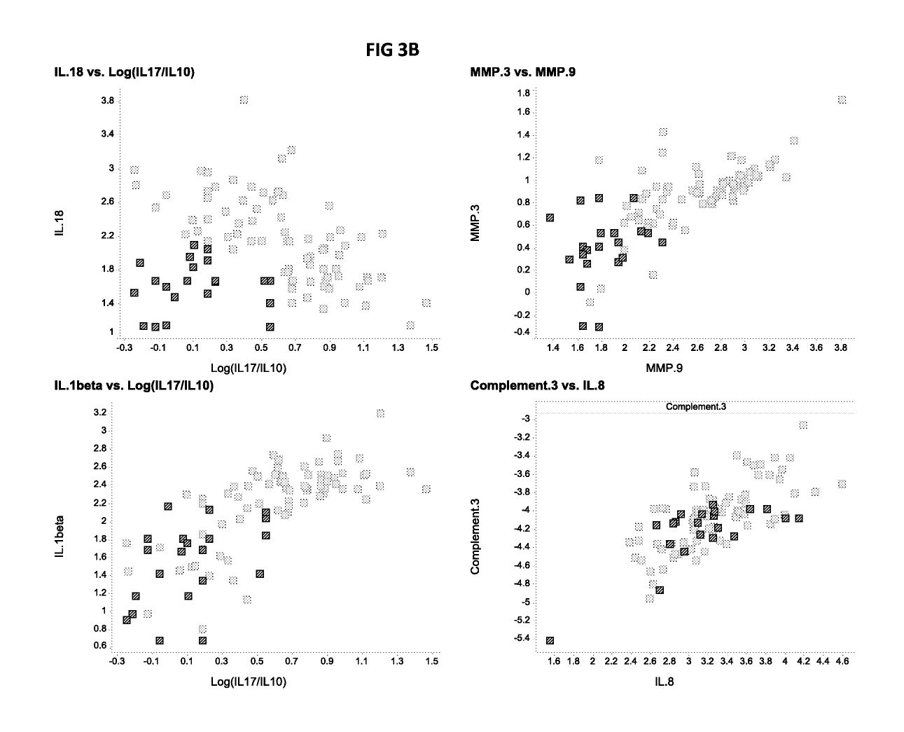

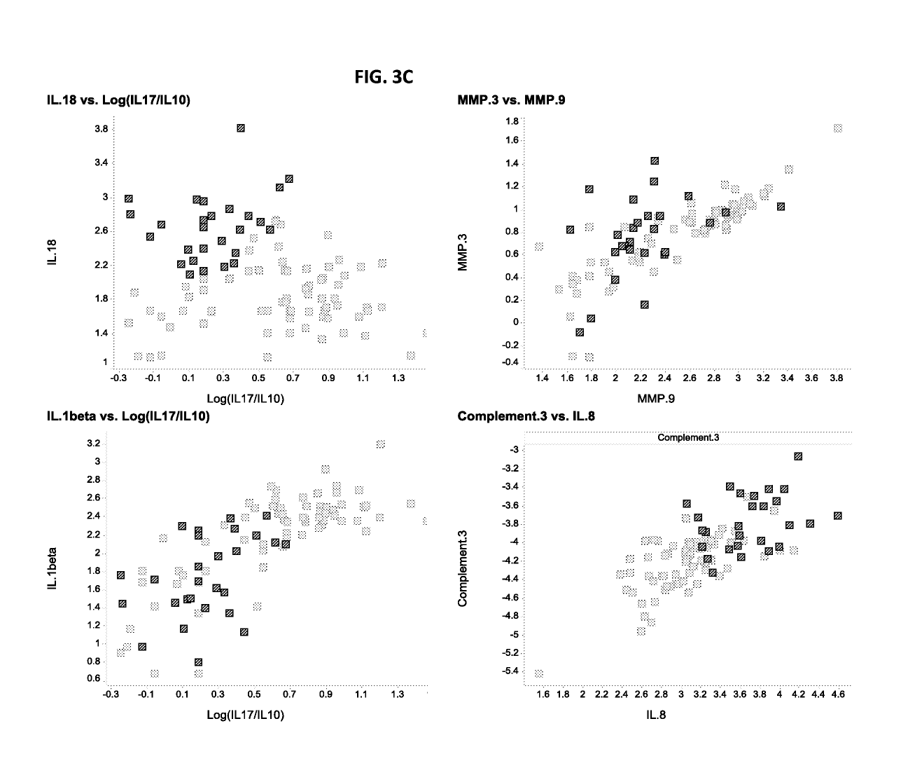

In a group of moderate-to-severe aqueous deficient dry eye patients, the tear level of IL-18 did not have strong correlation with IL-1 (r<0.30), IL-10 (r=0.35) or other Th2 associated cytokines; IL-18 only correlated weakly with IFN-gamma (r=0.53) and IL-2 (r=0.50); IL-1beta and IL-23 correlated well with IL-17. When plotting IL-18 versus IL-10 and IL-18 versus IL-17, more than one population could be identified, in particular, a group of patients having higher level of IL-17 than the rest of the subjects and they have higher level of IL-1beta and IL-23 also. See FIG. 3. Tear levels of IL-10 within this subgroup of patients were correlated positively with their levels of IL-18. Patients outside of this subgroup had high level of IL-18 but had low or absence of Th1 (IFNgamma and IL-2) and Th2 associated cytokines (IL-10, -5, -4, and -3). These results provided the evidence the balances normally present in homeostatic condition between IL-18 and IL-1 and between type 2 and type 17, and the association between IL-18 and type 2 cell immunity were altered in moderate-to-severe dry eye patients.

IL-1beta is generally produced by macrophages, IL-1alpha and IL-1Ra can be produced by epithelial cells. IL-18 could be produced by several types of cells including macrophages and epithelial cells. The production of IL-1 and IL-18 in control non-dry eye subjects may be regulated by the same mechanism, likely produced by resident macrophages. In moderate to severe dry eye patients, there may be multiple mechanisms and cell types that produce IL-18, depending on the patient subgroups, only one of them retain the correlation between IL-18 and IL-10 and other Th2 cytokines.

### 3) Disease Heterogeneity at Molecular Level: Distinct Patient Subgroups Classified Based on Underlying Molecular Mechanisms Using Biomarker Profiles or Levels of Several Biomarkers, Such as IL-1, IL-17, IL-10 and IL-18

In the group of moderate-to-severe aqueous deficient dry eye patients in the study, three main patient subpopulations could be identified: pathogenic IL-17 (with higher ratio of IL-17 over IL-10) group with higher level of IL-1beta and lower level of IL-18; two Non-T cell mediated subgroups (low levels of IL-17 and IL-10), one with lower level of IL-18 and IL-1beta, the other one with higher level of IL-18 and IL-1Ra. See FIG. 3.

These three subgroups in moderate to severe dry eye patients also exhibited differences in the tear levels of MMPs, complement 3 and IL-8: pathogenic IL-17 subgroup had highest level MMP-9 and MMP-3 but low level of complement 3 and IL-8; Non-T cell IL-18 low group had low levels of MMP-9 and -3, intermediate levels of complement and IL-8; Non-T cell IL-18 high group had intermediate levels of MMP-9 and -3, highest levels of complement and IL-8. See FIG. 3. These results provided evidence of distinct underlying mechanisms in different subgroups of patients within moderate-to-severe aqueous deficient dry eye patients. The molecular mechanisms and pathways of immunopathology include: IL-1 and IL-23 driven pathogenic IL-17, and high levels of MMP-9 and -3, leading to damages of the ocular surface epithelium and the ocular barrier breakdown; non-T cell driven, complement 3 and IL-8/neutrophil mediated, leading to microvascular leakages and ocular surface damages.

Pathogenic mechanism mediated by pathogenic Th17 cells is thought to be involved in several autoimmune conditions. The association of IL-18 and IL-1 Ra production by epithelial cells induced by TLR activation or type I IFN, and IL-8 production and complement activation induced by autoantibodies have been described in autoimmune conditions such as SLE and SS. Thus, heterogeneity in dry eye patients at underlying molecular level can be resulted from distinct mechanisms of immunopathology. They could be because of different pathogenic mechanisms in the disease, or different stages or status of the disease, or both. For example, patients in the Non-T cell driven IL-18 high group could have the same pathogenic mechanism as the Non-T cell driven IL-18 low, but they may have a fare up status during the time of the study, compared with patients in the Non-T cell driven IL-18 low.

### 5. Discussion

Analyzing immune mediators and protein biomarkers in tears obtained from DED patients and control subjects, distinct immunopathological mechanisms involved in dry eye disease were identified and found involved in different subgroups of dry eye patients. Thus, heterogeneity at contributing to patient heterogeneity at underlying mechanistic level in a group of moderate-to-severe aqueous deficient dry eye patients. into 3 otherwise undistinguishable subgroups corresponding to different underlying molecular mechanisms.

### 6. Conclusions

Biomarker profiles revealed distinct immunopathological mechanisms involved in autoimmune and inflammatory conditions, resulting in disease heterogeneity at molecular mechanistic level, in particular the roles of IL-1 versus IL-18, and the involvement of the two mechanisms: (1). IL-1, IL-23, pathogenic IL-17 and Th17 cells and MMP-9 and MMP-3 and (2) type I IFN, IL-18, IL-1Ra, IL-6, B cells, autoantibodies, IL-8/neutrophil, and complement. These different subtypes may also represent different stages of dry eye disease.

### Example 2

### Biomarker Profiles for Patient Heterogeneity at Underlying Molecular Mechanism Level in Dry Eye Patients

### 1. Introduction

Dry eye disease (DED) is one of the most prevalent ocular conditions in the US and many other regions in the world, affecting tens of millions of people worldwide. Ocular irritation and visual symptoms in DED, including ocular pain, gritty, sting and dryness in the ocular surface, fluctuated and blurred vision, often can significantly affect the quality of patients' daily life and work related activities. The term DED, also called tear-dysfunction, includes a heterogeneous group of conditions, each could associate with a different cause, such as systemic autoimmunity in Sjogren syndrome (SS) leading to ocular dryness, or obstruction of the meibomian gland in meibomian gland dysfunction (MGD) leading to changes in the composition and reduced quality of the tear film and ocular surface damage. All are characterized, however, by the development of ocular symptoms, accompanied by various clinical signs and pathological changes, including reduced tear production, unstable tear layer, irregular corneal surface, loss of goblet cells on the conjunctival epithelia, hyperplasia, and sensitization of corneal nerve endings and development of corneal epitheliopathy.

The etiology of dry eye disease is unknown. However, ocular surface inflammation mediated by CD4+ T cells is implicated in DED immunopathogenesis, based on observations of infiltration of CD4+ T cells and elevated level of proinflammatory mediators in the ocular surface in DED patients. Higher concentrations of cytokines and chemokines, including IL-1, IL-6, IL-8, TNF-a and MMP-9, were reported previously in tear fluids collected from patients with DED, using immunoassays. Increased RNA transcripts of genes encoding for IL-1b, TNF-a, IL-6, MMP-9 and chemokine receptor CCR5 were detected by real time PCR with conjunctival epithelial cells collected from patients with Sjogren syndrome or DED. Preclinical studies have implicated Th17 and Th1 cells in the pathogenesis of DED and shown the presence of dysfunctional Treg and the resistance of pathogenic T effector cells, in particular Th17 cells, to Treg suppression in experimental dry eye models. Blocking IL-17 with anti-IL-17 antibody ameliorated ocular surface pathology in experimental dry eye models.

However, significant heterogeneity of patient population in dry eye, limited correlation between different clinical parameters and poor reproducibility between clinical studies remain major challenges in dry eye clinical research and drug development.

### 2. Purpose

To identify and characterize the underlying molecular and cellular components involved in the immunopathology in dry eye, a large number of immune mediators and other protein factors of potential immunopathological relevance were profiled in tears from DED patients, and characterized potential underlying mechanisms and patient heterogeneity with multivariate systems biology approach.

### 3. Methods

Prospectively, at a screening visit in two investigator sites, 85 dry eye patients were recruited who had dry eye symptoms as measured with Ocular surface Disease Index (OSDI>=13) and different levels of corneal fluorescein staining (CS). Of these dry eye patients, 31 had CS score ranging from 0 to 3 (<4); 32 ranging from 4 to 6 (>=4 and <7); and 22 greater than 7 (>7). At Day 0 visit, 1-3 days after the Screening visit, and Day 7 visit, tear fluids were collected from each eye with micro-capillary tube and analyzed for 43 protein immune mediators or markers with potential immunopathological relevance by bead-based multiplexed immunoassay. In addition, at each visit, symptoms and clinical objective signs of DED were evaluated to assess disease characteristics.

### 4. Results

### 1) Correlations Between Protein Markers

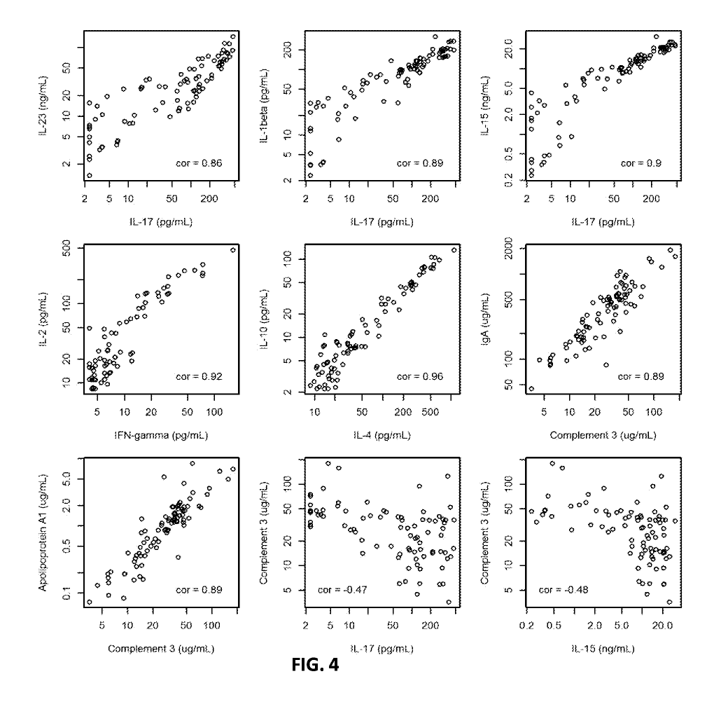

The pair-wise correlations were first examined between individual protein markers in tears collected at Day 0 visit by calculating the partial correlation coefficient (r) using regression analysis and controlling potential age effect. FIG. 4 shows pair-wise scatter plots of a few select markers that were correlated with each other.

Tear levels of IL-17A, IL-1b, IL-23 and IL-15 were strongly correlated with each other (r>0.85, P<0.0001). IL-17A is a Th17 cytokine produced by proinflammatory Th17 cells; IL-23 and IL-1beta, produced by dendritic cells and macrophages, are important for the polarization and proliferation of Th17 cells; IL-15 is thought to induce the production of IL-17A by Th17 cells. This group of cytokines and markers was termed the “IL-17 Cluster”. There were additional immune mediators correlated well (r>0.75, P<0.0001) with the IL-17 Cluster, including IL-12p70, Stem Cell Factor (SCF), Eotaxin and Factor 7.

Strong correlations were also observed between tear levels of IFNgamma (IFN-γ), IL-2 and TNFalpha (r>0.90, P<0.0001). IFN-γ is a prototypic Th1 cytokine, produced by proinflammatory Th1 cells, and it induces the production of TNFalpha. Similarly, strong correlations were also observed between tear levels of IL-4, -5, -3, -10, and GM-CSF (r>0.90, P<0.0001). Genes encoding for GM-CSF, IL-3, -4, -5 & -13 are localized to a cluster at chromosome region 5q31. IL-4, IL-5, IL-13 and IL-10 are considered Th2 associated cytokines, and Th2 is primarily involved in allergy. (IL-13 was not assayed in this study). These two groups of cytokines were termed “Th1 Cluster” and “Th2 Cluster”, respectively. Interestingly, in this study, cytokines of Th1 and Th2 Clusters were strongly correlated with each other as well (r>0.85, P<0.0001), but had much weaker correlation with cytokines of IL-17 Cluster (r<0.65). For example, the correlation was 0.91 between IL-5 and IFNg, but was only 0.56 between IL-17A and IFNalpha, and 0.67 between IL-17A and IL-5. IL-18 was also correlated well with the Th1 Cluster (r>0.80, P<0.0001).

Tear level of IL-18 did not have strong correlations with IL-1 (r<0.30), IL-10 (r=0.35) or other Th2 associated cytokines; IL-18 only correlated weakly with IFN-gamma (r=0.53) and IL-2 (r=0.50); on the other hand, IL-1beta and IL-23 correlated well with IL-17.

Tear levels of Complement 3 (C3), IgA, IgM, Alpha-1 antitrypsin (AAT), and Apolipoprotein (Apo) H, A1 and CIII correlated well with each other (r>0.80, P<0.0001), and thus named “C3 Cluster”.

These strong positive correlations between markers indicated that their productions were interdependent, co-regulated, or associated with a common source, such as a particular cell type in the ocular surface of dry eye patients.

Interestingly, the IL-17 Cluster was found to have an inverse correlation, although weak, with C3 Cluster: r ranged from −0.48 to −0.44 between C3 and IL-15, IL-17A and IL-1beta (FIG. 4).

### 2) Clustering Analysis of Patients

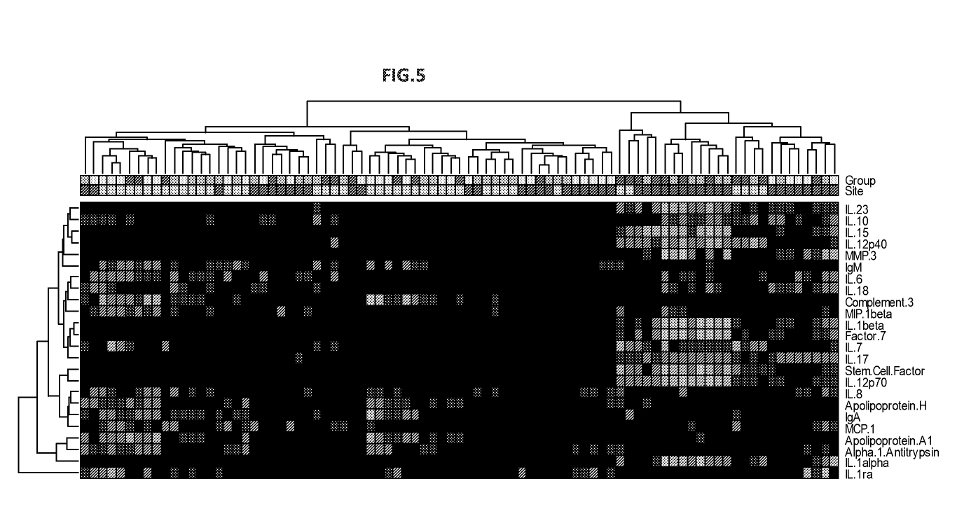

An unsupervised approach was then used to analyze patients based on their tear marker profiles, after excluding markers that were below detection limit in more than 25% of tear samples (12 markers) or showed low variability across samples (7 markers). Many of the Th1 and Th2 cytokines including IFNgamma, IL-2, TNFalpha, IL-5 and IL-3 were among the markers excluded from this analysis because of low levels in more than 25% of samples. Remaining twenty four markers were used in the hierarchical clustering analysis and 2 distinct patient groups were apparent within the dataset, one with 60 patients and the other with 25. Each group could be further divided into 2 subgroups: 31 and 29 patients in subgroup 1 and 2, 5 and 20 patients in subgroup 3 and 4, respectively. See FIG. 5. Notably, these patient subgroups were detectable even if different clustering algorithms were used (average linkage or complete linkage). These subgroups of patients were not associated with individual investigator sites or dry eye patient groups prospectively defined at screening visit. See FIG. 5. Similar pattern was observed when clustering analysis was conducted using either study eye from each patient (prospectively defined as the worst eye based on corneal staining at Screening visit), or taking the average of the two fellow eyes. Consistent with DED being a bilateral ocular condition, both clinical parameters and many tear cytokines and protein markers are comparable between fellow eyes. Thus, as described in FIG. 5 and elsewhere herein, the analysis was based on taking the average of the two fellow eyes of each subject.

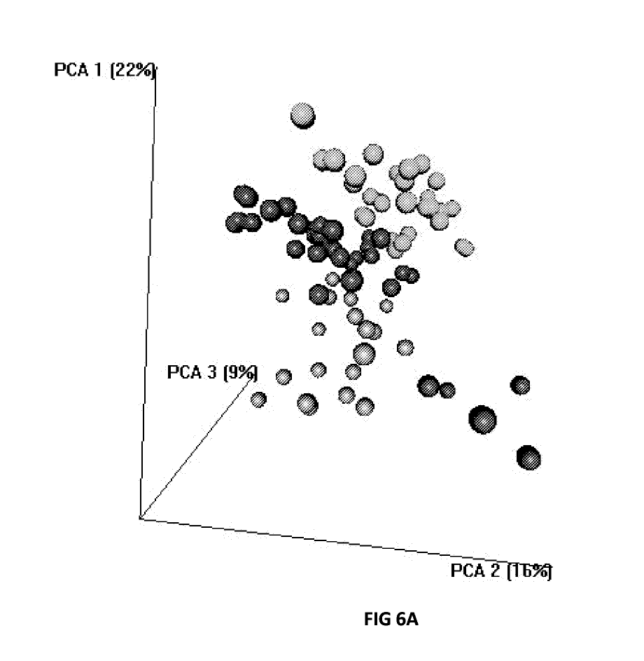

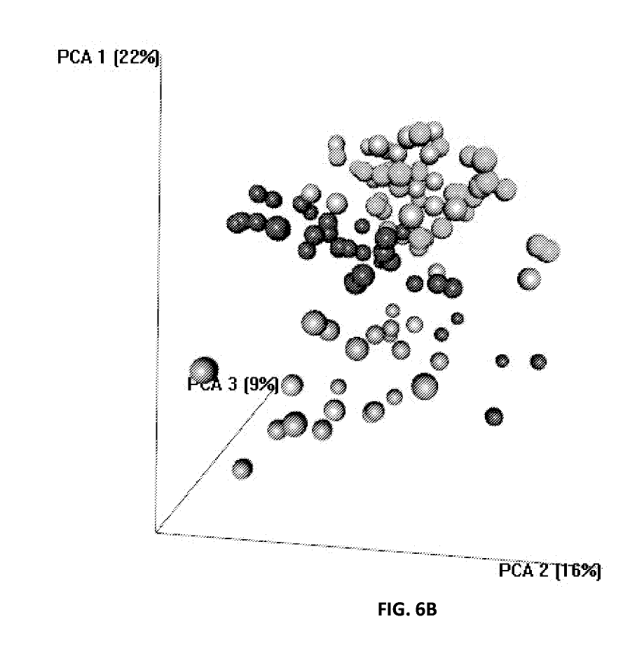

These subgroups of patients revealed by clustering analysis were also distinguishable by principal component analysis (PCA) of the 85 dry eye patients based on their tear marker profiles. See FIG. 6.

### 3) Differential Expression of Tear Protein Markers Among Subgroups

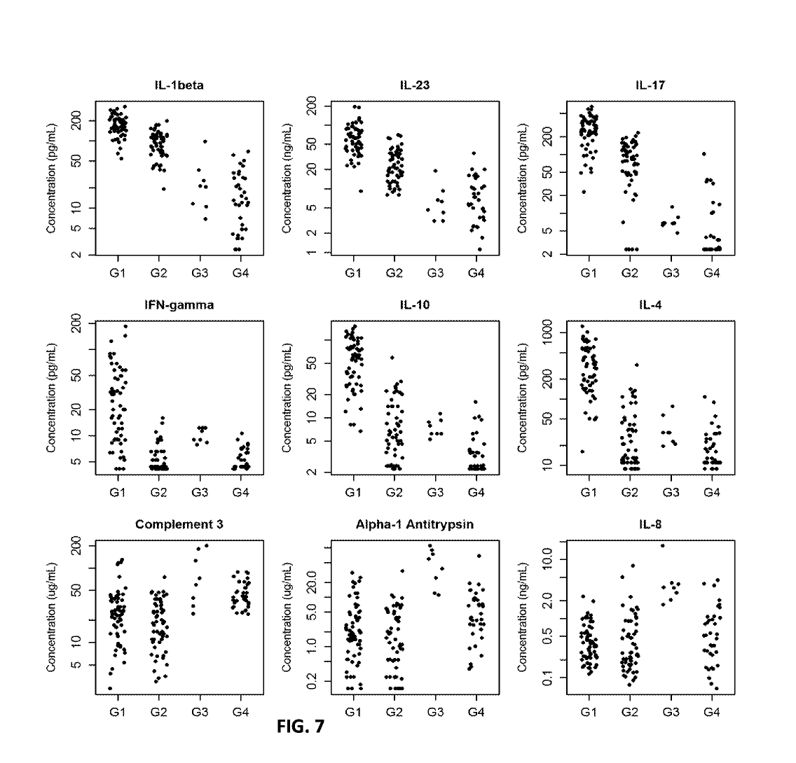

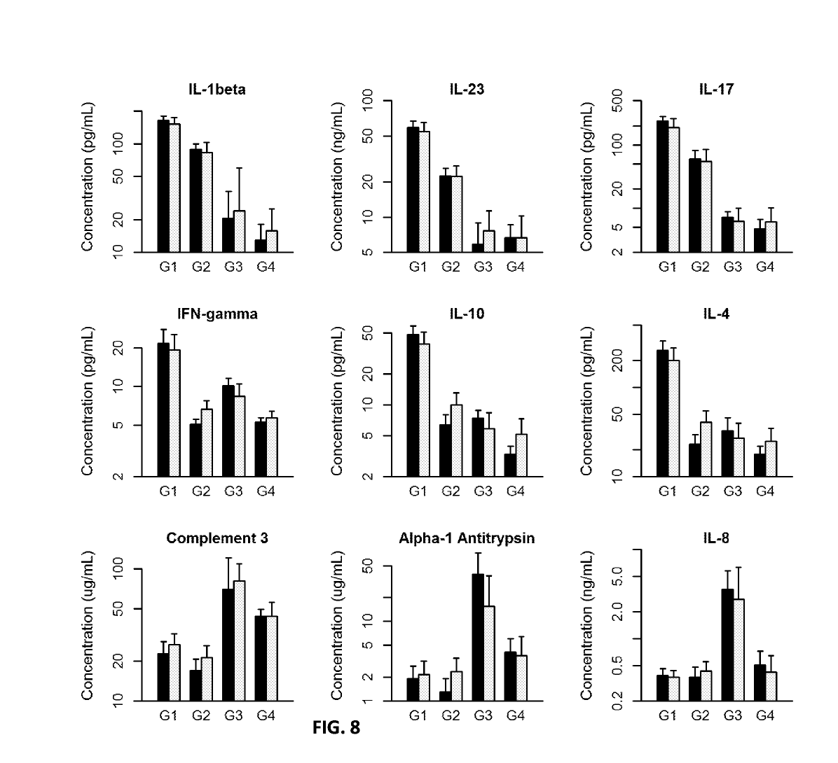

Next, differential expression of the protein markers was examined among the four subgroups of patients identified from the clustering using analysis of covariance (ANCOVA) adjusting for the potential effect of age, gender and study sites. P values from post hoc pair-wise comparisons were corrected for multiple hypothesis testing. Select cytokines and immune mediators are shown in FIG. 7.

Tear levels of cytokines in the IL-17 Cluster in patients of Subgroups 1 and 2 were significantly higher than in Subgroups 3 and 4 (p<0.0001). They were the highest in Subgroup 1 and significantly higher than the ones in Subgroup 2 (p<0.0001); while they were similar between Subgroups 3 and 4 (p>0.05). Patients were thus categorized in Subgroups 1 as IL-17high, Subgroups 2 as IL-17intermediate and the ones in Subgroup 3 and 4 as IL-17low. As shown in FIG. 4, the mean concentration of IL-17 in Subgroup 1 (236.26 ug/mL, 95% CI: 199.25-280.15) was nearly 4 fold higher than that in Subgroup 2 (59.59 ug/mL, 95% CI: 43.63-81.39, p<0.0001), 33 fold of Subgroup 3 (7.12 ug/mL, 95% CI: 5.82-8.71, p<0.0001) and 50 fold of Subgroup 4 (4.7 ug/mL, 95% CI: 3.33-6.62, p<0.0001). It was 8 and 12 fold higher in Subgroup 2 than in Subgroup 3 and 4 (p<0.0001), but similar between Subgroup 3 and 4 (p=0.76).

Tear level of cytokines in Th1 and Th2 Clusters was significantly higher only in patients of Subgroup 1, but not in Subgroups 2, 3 or 4 (P<0.0001) (FIG. 7), different from cytokines of IL-17 Cluster. Thus, patients in Subgroup 1 were categorized as IL-17high/Th1Th2high while ones in Subgroup 2 as IL-17intermediate/Th1Th2low. Conversely, tear levels of many markers in C3/Serum Cluster were found to be highest in Subgroup 3, followed by Subgroup 4, and lowest in Subgroups 1 and 2. As shown in FIG. 7, the mean level of C3 was significantly higher in Subgroup 3 (69.83 ug/mL, 95% CI: 40.15-121.46) than in Subgroup 1 (22.9 ug/mL, 95% CI: 18.58-28.23, p=0.0004), Subgroup 2 (17.01 ug/mL, 95% CI: 13.92-20.79, p<0.0001), and Subgroup 4 (43.85 ug/mL, 95% CI: 38.71-49.68, p=0.065); it was also significantly higher in Subgroup 4 than in Subgroup 2 (p=0.0002) and Subgroup 1 (p=0.0351). Patients in Subgroups 3 and 4 were thus categorized as C3high and C3intermediate, respectively.

Interestingly, tear level of IL-8 (CXCL-8) was also significantly higher in patients of Subgroup 3 (3.59 ng/mL, 95% CI: 2.22-5.8) than those of Subgroup 1 (0.39 ng/mL, 95% CI: 0.32-0.46), 2 (0.37 ng/mL, 95% CI: 0.28-0.48) or 4 (0.51 ng/mL, 95% CI: 0.35-0.72) (p<0.0001). See FIG. 7.

To summarize, using unsupervised clustering analysis of the tear cytokine profiling data, DED patients in this study were found to segregate into four subgroups with distinct molecular profiles: IL-17high/Th1Th2high/C3low (Subgroup 1), IL-17intermediate/Th1Th2low/C3low (Subgroup 2), IL-17low/Th1Th2low/C3high/IL-8high (Subgroup 3) and IL-17low/Th1Th2low/C3intermediate/IL-8low (Subgroup 4).

### 4) Tear Marker Profiles Stable Over a Week

These differential expression profiles of tear cytokines in DED patients revealed by clustering with dataset from Day 0 visit were highly stable since concentrations of markers detected on Day 7 visit were similar to those on Day 0. FIG. 5 shows group mean concentrations and 95% Cl of select protein markers in the 4 patient subgroups at Day 0and Day 7.

### 5) Clinical Characteristics of Patient Subgroups

Patient demographics were next examined and compared dry eye clinical parameters among the four patient subgroups using ANCOVA to adjust for the potential effect of age, gender and study sites. P values from post-hoc pair-wise comparisons between subgroups were adjusted for multiple testing. The group mean and standard deviation of each subgroup and P values are listed in Table 2, below.

On average, patients in Subgroup 3 had the worst clinical features, followed by those in Subgroup 4: higher corneal and conjunctival staining, lower STT wetting and TBUT, and the worst symptoms measured by OSDI and Ocular comfort index (OCI). Specifically, corneal and conjunctival staining were significantly worse in Subgroup 3 than in Subgroups 1 (P<0.0001), 2 (p<0.0001) and 4 (p<0.01), while they were not significantly different between Subgroup 1, 2 and 4. STT without anesthesia was lower in Subgroup 3 and 4 than in Subgroup 1 and 2 (p<0.05). Patients in Subgroup 4 have slightly worse STT wetting than Subgroups 1 (p=0.006) and 2 (p=0.016), and higher conjunctival staining than subgroup 1 (p=0.001). The only significant difference found between Subgroup 1 and 2 was TBUT, shorter (less protection for the ocular surface) in Subgroup 1 than in Subgroup 2 (p=0.003).

On average, patients in Subgroup 3 had the worst clinical features, followed by those in Subgroup 4: higher corneal and conjunctival staining, lower STT wetting and TBUT, and the worst symptoms measured by OSDI and Ocular comfort index (OCI). Specifically, corneal and conjunctival staining were significantly worse in Subgroup 3 than in Subgroups 1 (P<0.0001), 2 (p<0.0001) and 4 (p<0.01), while they were not significantly different between Subgroup 1, 2 and 4. STT without anesthesia was lower in Subgroup 3 and 4 than in Subgroup 1 and 2 (p<0.05). Patients in Subgroup 4 have slightly worse STT wetting than Subgroups 1 (p=0.006) and 2 (p=0.016), and higher conjunctival staining than subgroup 1 (p=0.001). The only significant difference found between Subgroups 1 and 2 was TBUT, shorter (less protection for the ocular surface) in Subgroup 1 than in Subgroup 2 (p=0.003).

### 5. Discussion

This example shows the identification in the tear film of dry eye patients the presence of protein biomarker signatures of IL1b/IL-23-IL-17A cascade, Complement 3/serum proteins, and IL-8/neutrophil. These biomarker signatures indicate the infiltration and activation of DCs, macrophages, and IL-17 producing cells, microvascular leakage, and infiltration of neutrophils, respectively, in the ocular surface in DED. These tear marker profiles revealed considerable patient heterogeneity at molecular mechanistic level and biomarker signatures could differentiate and stratify patients into at least 2-3 but otherwise undistinguishable subsets. One subset of DED patients have high level of IL-17A, IL-1, IL-12, IL-15, IL-23 and MMP-9 in their tear fluid, indicating activation of IL-1b/IL-23/IL-12 producing DCs and macrophages, and infiltration and activation of IL-17A producing cells at the ocular surface (IL-1beta^{high}/IL-23^{high}/IL-12^{high}/IL-17A^{high}); while another subset is associated with suppressed levels of IL-1beta, IL-23 and IL-17A, but elevated level of Complement 3 (IL-1beta^{low}/IL-23^{low}/IL-17A^{low}/C3^{high}), a potential indication of presence of autoantibody, Within this group of IL-17 low and C3 high patients, a subset of them have significantly elevated tear level of IL-8 and serum proteins (AAT, apolipoprotein A1 and H) (IL-1b^{low}/IL-23^{low}/IL-17A^{low}/C3^{high}/IL-8^{high}/AAT^{high}), indication of inflammation induced vascular dilation and plasma exudation.

TABLE 2
Patient demographics and dry eye clinical characteristics in each of the subgroups at Day 0 visit.

| | G1 | G2 | G3 | G4 | P | | | | | | |
|---|---|---|---|---|---|---|---|---|---|---|---|
| | (n = 31) | (n = 29) | (n = 5) | (n = 20) | Overall | G2-G1 | G3-G1 | G4-G1 | G3-G2 | G4-G2 | G4-G3 |
| Age (yr) | 46.7 | 47.9 | 56.2 | 51.8 | 0.457 | | | | | | |
| (range) | (19-85) | (18-88) | (44-70) | (26-70) | | | | | | | |
| Female/ | 24/7 | 18/11 | 2/3 | 14/6 | 0.174 | | | | | | |
| Male | | | | | | | | | | | |
| Corneal | 4.3 | 4.2 | 8.5 | 5.2 | <0.0001 | 0.994 | <0.0001 | 0.231 | <0.0001 | 0.167 | 0.003 |
| Staining | (0.32) | (0.29) | (0.76) | (0.4) | | | | | | | |
| (NEI) | | | | | | | | | | | |
| Conjunctival | 0.9 | 1.5 | 3.4 | 1.6 | <0.0001 | 0.142 | <0.0001 | 0.001 | <0.0001 | 0.132 | 0.005 |
| Staining | (0.13) | (0.19) | (0.92) | (0.27) | | | | | | | |
| (Oxford) | | | | | | | | | | | |
| STT. w/o | 18.9 | 17.9 | 6.1 | 11.1 | 0.001 | 0.991 | 0.038 | 0.006 | 0.051 | 0.016 | 0.843 |
| anesthesia | (1.49) | (1.53) | (0.93) | (1.31) | | | | | | | |
| (mm) | | | | | | | | | | | |
| STT. w/ | 16.9 | 18.7 | 7.3 | 16.2 | <0.0001 | 0.166 | 0.036 | 0.378 | 0.001 | 0.007 | 0.277 |
| anesthesia | (1.28) | (1.27) | (1.53) | (1.8) | | | | | | | |
| (mm) | | | | | | | | | | | |
| TBUT | 3.4 | 4.3 | 2.9 | 3.4 | 0.004 | 0.003 | 0.992 | 0.452 | 0.197 | 0.440 | 0.696 |
| (second) | (0.18) | (0.16) | (0.41) | (0.28) | | | | | | | |
| Tear | 304.0 | 300.8 | 305.5 | 305.7 | 0.058 | 0.572 | 0.708 | 0.417 | 0.327 | 0.054 | 0.997 |
| Osmolarity | (2.04) | (1.25) | (3.24) | (1.82) | | | | | | | |
| AT dose | 2.2 | 2.4 | 2.8 | 3.3 | 0.101 | 0.769 | 0.774 | 0.061 | 0.966 | 0.381 | 0.950 |
| | (0.26) | (0.28) | (0.33) | (0.44) | | | | | | | |
| OSDI | 43.0 | 41.5 | 50.5 | 47.47 | 0.198 | 0.994 | 0.513 | 0.397 | 0.430 | 0.302 | 0.962 |
| | (2.25) | (2.51) | (10.68) | (3.14) | | | | | | | |
| NEI-VFQ-25 | 71.38 | 73.74 | 74.67 | 73.91 | 0.695 | 0.663 | 0.967 | 1.000 | 1.000 | 0.833 | 0.979 |
| | (1.99) | (2.14) | (4.37) | (1.94) | | | | | | | |
| OCI | 42 | 45.56 | 52.27 | 46.77 | 0.013 | 0.096 | 0.025 | 0.226 | 0.290 | 1.00 | 0.294 |
| | (1.15) | (1.75) | (2.69) | (2.01) | | | | | | | |

NEI: National Eye Institute grading scale; Conjunctival Staining: Lissamine green using Oxford scale; STT. w/o anesthesia: Schirmer's tear test without anesthesia. STT. w/ anesthesia: Schirmer's tear test with anesthesia; TBUT: Tear breakup time. P value: overall p value for group comparison from ANCOVA analysis adjusting for the effect of age, gender and site, and corrected for multiple comparisons. AT dose; average artificial tear eye drop dose recorded by patient diary. OSDI: Ocular Surface Disease Index. NEI-VFQ-25: National Eye Institute Visual Function Questionnaires-25. OCI: Ocular Comfort Index.

### 6. Conclusions

This study suggests that dry eye is a heterogeneous disorder with distinct underlying immunopathological mechanisms, and tear biomarkers could be used to diagnose and differentiate the different molecular mechanisms in dry eye patients, thus stratify the disease (patients) for better diagnosis, monitoring and treatment selection.

In this study, multiple subgroups of dry eye patients were identified based on the profiles of protein biomarkers found in tear fluid. Different subgroups of patients could be segregated by distinct molecular components of immune activation, implicating different immunopathological mechanisms involved in patients with ocular surface disease. While not wishing to be bound to any particular theory, one of these mechanisms involves IL-1 and is proinflammatory, T cell driven. Another, which has not been recognized before in human DED, is associated with complement activation, and is IL-8 driven and non-T cell-mediated. These different DED subgroups may also represent different stages of dry eye disease, which could explain the discordance observed between clinical signs and patient-reported DED symptoms. Classifying (or stratifying) DED into biomarker signature-based subgroups (alone or in conjunction with symptomatic and/or clinically defined symptoms) will be useful in the selection of patient populations for targeted therapy, as well as in the selection of clinically appropriate endpoints to mitigate the risks of significant inconsistencies currently experienced in programs aimed at developing drugs to treat DED.

### Example 3

Confirmation of Biomarker Signatures Associated with Multiple Distinct Immunopathological Mechanisms that Contributing to Patient Heterogeneity in Moderate-to-Severe Aqueous Deficient Dry Eye Patients

### 1. Introduction

This example describes a study that involved identification and characterization of the underlying macular and cellular components that contribute to the heterogeneity of ocular surface disease pathology and manifestation in dry eye disease. In this study, patients with moderate-to-severe aqueous deficient dry eye disease were characterized at the molecular level by tear protein marker profiling.

### 2. Purpose

The term dry eye disease, also called tear-dysfunction, includes a heterogeneous group of ocular surface conditions each associated with a different cause, such as systemic autoimmunity in Sjogren syndrome (SS) leading to ocular dryness, or obstruction of the meibomian gland in meibomian gland dysfunction (MGD) leading to changes in the composition and reduced quality of the tear film and ocular surface damage. All are characterized, however, by the development of ocular symptoms, accompanied by various clinical signs and pathological changes, including reduced tear production, unstable tear layer, irregular corneal surface, loss of goblet cells on the conjunctival epithelia, hyperplasia and sensitization of corneal nerve endings, and development of corneal epitheliopathy.

According to the report of Dry Eye WorkShop (DEWS), DED is classified into two major types: aqueous-deficient and evaporative dry eye. Aqueous deficient dry eye includes SS (primary and secondary) and non-SS aqueous deficient dry eye. SS is a systemic autoimmune disease affecting the lachrymal and salivary glands, and can develop alone or in association with other autoimmune diseases such as systemic lupus erythematosus (SLE) and rheumatoid arthritis (RA). In SS dry eye patients, destruction and malfunction of the lachrymal gland, the exocrine gland responsible for secreting aqueous component of the tear film in humans, lead to significant reduction in tear production and ocular dryness. The majority of aqueous dry eye patients are non-SS dry eye with unknown etiology. They have dry eye symptoms, reduced tear production, and other ocular clinical signs but have no apparent underlying systemic diseases. One of the common causes of evaporative dry eye is MGD, in which inflammation and obstruction of the meibomian gland can lead to alterations in the normal lipid composition in meibomian gland secretions, therefore leading to abnormalities of tear film composition and function, resulting in increased tear breakup time (TBUT), ocular surface damage, and dry eye symptoms. Although both aqueous deficient and evaporative dry eye are common, there are considerable overlaps of both types in many clinical DED patients. In this separate multicenter dry eye clinical study, moderate-to-severe aqueous deficient dry eye patients selected with a different set of inclusion and exclusion criteria were examined at the molecular level by analyzing tear protein marker profiles and to assess if similar molecular profiles could be identified for disease heterogeneity.

### 3. Methods

Study Design and Patients. Fifty-nine moderate to severe aqueous deficient DED patients age 18 years or older with a diagnosis of DED for at least 6 months were enrolled in three clinical centers. They met a set of inclusion and exclusion criteria designed for selecting moderate-to-severe aqueous deficient DE patients, specifically, Schirmer wetting test without anesthesia: ≧1 mm and ≦7 mm/5 min; Corneal fluorescein staining total score of ≧4 (NEI), and a score of at least 3 (0-6 scale) on 4 of the 16 questions in the Ocular Comfort Index (OCI), which asks patients to rate the frequency and intensity of eight common DED symptoms (ocular dryness, grittiness, stinging, fatigue, pain, itching, blurred vision, and photophobia).

At the baseline visit, after 2 weeks of wash-out period, non-stimulated tear fluids were collected from each eye and analyzed for 43 protein biomarkers, in the same way as described previously. In addition, symptoms and clinical objective signs of DED were evaluated, including corneal fluorescein staining (NEI), lessimineconjunctival staining (Oxford scale), Schirmer's test conducted with and without anesthesia, tear breakup time (TBUT).

Statistical Analysis. Protein markers that were below the least detectable dose (LDD) or had missing values in more than 25% tear samples or showed low variability (coefficient of variation [CV]<10%) across samples were excluded from unsupervised clustering analysis. Two-way hierarchical clustering based on Euclidean distance using average linkage was performed for both patients and protein markers. Analysis of covariance (ANCOVA) including age, gender, and clinical sites as covariates was used to assess if the levels of tear protein markers differ among subgroups of patients identified by clustering analysis. Post-hoc pairwise comparisons among patient subgroups were performed by general linear hypothesis and p values were estimated with multiplicity adjustment. Statistical significance was set at 0.05; all p values were 2-sided.

### 4. Results

Similarly good correlations were found between cytokines within the IL-17 Cluster and between proteins within C3 Cluster (r>0.80, P<0.0001). Tear levels of IL-17A, IL-23, IL-1b, IL-15 and MMP-9 correlated well with each other (r>0.80, P<0.0001); so were between C3, AAT, apo A1 and other serum proteins (r>0.85, P<0.0001). Tear level of IL-8 also correlated well with AAT (r>0.80, P<0.0001) and other plasma proteins in this study.

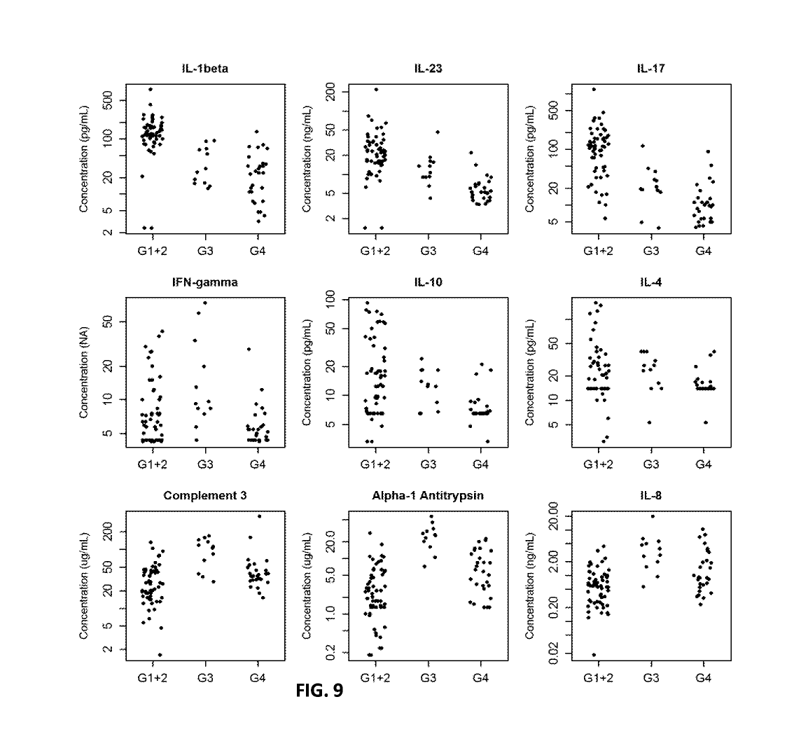

Using heretical clustering analysis, the 59 patients were clustered into 3 groups and the differential expression of the markers among the 3 groups was examined using ANCOVA adjusting for the potential effect of age, gender and study sites, with P values from post-hoc comparisons corrected for multiple analyses. 32 patients clustered into one group which had significantly higher level of cytokines of IL-17 Cluster, lower level of IL-8 and markers in C3 Cluster than the other two groups (P<0.05) (IL-17high/C3low/IL-8low). This profile is similar to that of patients in Subgroups 1 and 2 in the first study, since the group means of Th1 and Th2 cytokines were not significantly different from the other two subgroups of patients, thus this was termed Subgroup 1/2 (FIG. 9). The second group of patients (N=9) revealed by clustering exhibited characteristics of Subgroup 3 in the first study, in particular having the highest level of IL-8 and proteins in the C3 Cluster (P<0.05) (IL-17low/C3high/IL-8high). The third group of patients (N=18) was found to have a profile similar to the Subgroup 4 previously: lowest level of IL-17A, IL-23, IL-1a, MMP-3 and MMP-9 (P<0.01), and intermediate level of IL-8, C3 and AAT (P<0.05) (IL-17low/C3intermediate/IL-8intermediate) (FIG. 9).

The dry eye clinical signs and symptoms between the three groups of patients were compared using ANCOVA (Table 2). Unlike the first study, dry eye clinical signs: CS, conjunctival staining, Schirmer I test, and TBUT, were not significantly different between patient groups (P>0.05); OSDI symptom score was worse in patients of IL-17high/C3low/IL-8low than those of IL-17low/C3intermediate/IL-8intermediate (p<0.001). It was noted that the mean OSDI score of patient groups of IL-17low/C3high/IL-8high (26.63±2.65) and IL-17low/C3intermediate/IL-8intermediate (27.81±2.33) in the second clinical study was lower than the ones in the first clinical study (50.5±10.68; 47.47±3.14), while the mean OSDI score of patient group of IL-17high/C3low/IL-8low (48.86±3.3) was more comparable to those in the first study (43±2.25; 41.5±2.51). See Table 3, below.

TABLE 3
Dry eye clinical signs and symptoms in each subgroup of patients in a separate
independent study in moderate to severe aqueous deficient dry eye patients.

| | | | | | P Value | | |
|---|---|---|---|---|---|---|---|
| | Mean | | | | G3- | G4- | |
| | G(1 + 2)(n = 32) | G3(n = 9) | G4(n = 18) | Overall | G(1 + 2) | G(1 + 2) | G4-G3 |
| Corneal | | | | | | | |
| Staining | 5.04 (0.17) | 5.08 (0.34) | 5.32 (0.35) | 0.741 | 0.980 | 0.716 | 0.940 |
| Conj. Staining | 1.58 (0.18) | 0.75 (0.22) | 1.5 (0.3) | 0.446 | 0.516 | 0.957 | 0.414 |
| STT. w/o | 6.18 (0.45) | 6.25 (0.94) | 6.86 (0.92) | 0.687 | 0.999 | 0.680 | 0.853 |
| STT. w/ | 8.7 (0.62) | 16.3 (2.35) | 12.0 (1.21) | 0.001 | 0.001 | 0.076 | 0.086 |
| TBUT | 3.92 (0.1) | 3.56 (0.18) | 3.88 (0.12) | 0.113 | 0.090 | 0.802 | 0.220 |
| OSDI | 48.86 (3.3) | 26.63 (2.65) | 27.81 (2.33) | <0.001 | 0.127 | <0.001 | 0.744 |
| OCI | 47.44 (1.15) | 49.52 (1.77) | 43.89 (1.38) | 0.071 | 0.449 | 0.285 | 0.074 |

STT. w/o anesthesia: Schirmer's tear test without anesthesia. STT. w/ anesthesia: Schirmer's tear test with anesthesia; TBUT: Tear breakup time. P value: overall p value for group comparison from ANCOVA analysis adjusting for the effect of age, gender and site, and corrected for multiple comparisons. OSDI: Ocular Surface Disease Index. OCI: Ocular Comfort Index.

### 5. Conclusion

A different tear protein marker dataset from a separate clinical study conducted in moderate to severe aqueous deficient dry eye patients was analyzed and it was confirmed that similar molecular profiles could be identified with tear biomarkers, confirming the consistent involvement of these immunopathological mechanisms in dry eye patients and the utility of the tear biomarkers and biomarker signatures.

All of the compositions, articles, devices, systems, and methods disclosed and claimed herein can be made and executed without undue experimentation in light of the present disclosure. While the compositions, articles, devices, systems, and methods of this invention have been described in terms of preferred embodiments, it will be apparent to those of skill in the art that variations may be applied to the compositions, articles, devices, systems, and methods without departing from the spirit and scope of the invention. All such variations and equivalents apparent to those skilled in the art, whether now existing or later developed, are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each individual publication was specifically and individually indicated to be incorporated by reference in its entirety for any and all purposes.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims, which may also contain even further embodiments of the invention.

What is claimed is:

1. A diagnostic method for differentiating underlying immunopathological mechanisms in patients having an autoimmune or inflammatory condition, comprising:
a. contacting a biological sample, optionally a tear sample, obtained from a subject known to have or suspected of having autoimmune or inflammatory disease with a plurality of detection reagent species that each independently binds a biomarker species associated with an immunopathological mechanism involved in an autoimmune or inflammatory disease different from biomarker species bound by the other detection reagent species; and
b. using the detection reagent species to determine if the biomarker species associated with the immunopathological mechanism in patients having an autoimmune or inflammatory disease are present in amounts indicative of an autoimmune or inflammatory condition, optionally, dry eye disease.

2. A method according to claim 1 wherein the biomarker species include at least one biomarker species selected from the group consisting of IL-1beta, IL-1alpha, IL-1Ra, IL-15, IL-7, IL-2, IL-3, IL-4, IL-5, IL-6, GM-CSF, IL-18, IL-8, IL-12p70, IL-12p40, IL-17, IL-23, CXCL-10, ICAM-1, MIP-1alpha, MIP-1 beta, Complement 3, alpha1-antitrypsin, apolipoprotein A1, apolipoprotein CIII, and IgM, and/or any of derivative or fragment of any of the foregoing.

3. A method according to claim 1 wherein at least one, some, or all of the detection reagent species comprise an antibody or antigen-binding antibody fragment.

4. A method according to claim 3 wherein the method comprises performing a multiplex immunoassay.

5. A method according to claim 1 wherein the method is used for diagnosis, patient stratification, to monitor the progression or status of autoimmune or inflammatory disease in the subject, or to monitor the efficacy of a therapy to treat autoimmune or inflammatory disease.

6. A method according to claim 1 wherein the amount of at least one of the biomarker species is associated with an immunopathological mechanism involved in an autoimmune or inflammatory disease is a measured concentration, and the determining step comprises comparing the concentration to a threshold concentration for the biomarker, wherein, depending upon the biomarker, a measured concentration substantially equal to or greater than or less than the threshold concentration for the biomarker is indicative of an immunopathological mechanism involved in an autoimmune or inflammatory condition i.

7. A method according to claim 1 that comprises use of at least three different detection reagent species.

8. A diagnostic kit for assessing an immunopathological mechanism involved in an autoimmune or inflammatory disease, comprising:
a. a plurality of detection reagent species that each independently bind a biomarker species associated with an immunopathological mechanism involved in an autoimmune or inflammatory condition, optionally dry eye disease, different from biomarker species bound by the other detection reagent species in the kit; and
b. instructions for using the detection reagent species to analyze a biological sample, optionally a tear sample, obtained from a subject to determine if the sample contains an amount of the biomarker species indicative of an immunopathological mechanism involved in an autoimmune or inflammatory condition.

9. A kit according to claim 8 wherein at least one of the detection reagent species comprises an antibody or antigen-binding antibody fragment.

10. A kit according to claim 8 wherein the detection reagent species are immobilized on one or more solid substrates.

11. An immunopathological mechanisms diagnostic method, comprising:
a. contacting a biological sample, such as a tear sample, obtained from a subject known to have or suspected of having an autoimmune or inflammatory condition with a plurality of detection reagent species that each independently bind a biomarker species associated with an autoimmune or inflammatory disease different from biomarker species bound by the other detection reagent species;
b. using the plurality of detection reagent species to determine whether a biomarker signature indicative of an immunopathological mechanism is present in the biological sample, and, if a biomarker signature indicative of an immunopathological mechanism is present, optionally determining which biomarker signature is present in order to classify which immunopathological mechanism afflicts the subject from whom the biological sample was obtained.

12. A method according to claim 11 wherein the biomarker species include at least one biomarker species selected from the group consisting of IL-1beta, IL-1alpha, IL-1Ra, IL-7, IL-2, IL-3, IL-4, IL-5, IL-6, GM-CSF, IL-18, IL-8, IL-12p70, IL-12p40, IL-17, IL-23, CXCL-10, ICAM-1, MIP-1alpha, MIP-1beta, MMP-3, MMP-9, Complement 3, alpha1-antitrypsin, apolipoprotein A1, apolipoprotein CIII, and IgM, and/or any of derivative or fragment of any of the foregoing.

13. A method according to claim 11 wherein at least one, some, or all of the detection reagent species comprise an antibody or antigen-binding antibody fragment.

14. A method according to claim 13 wherein the method comprises performing a multiplex immunoassay.

15. A method according to claim 11 wherein the method is used for patient stratification, treatment selection, to monitor the progression or status of an autoimmune or inflammatory condition in the subject, or to monitor the efficacy of a therapy to treat an autoimmune or inflammatory condition, optionally, dry eye disease.

16. A method according to claim 11 wherein the determining step comprises comparing the biomarker signature to a threshold signature, wherein, depending upon the biomarker species that comprise the biomarker signature, a biomarker signature substantially equivalent to or different than the threshold signature is indicative of an underlying immunopathological mechanism.
